SEQUENCE LISTING (6 sequences)

SEQ ID NO: 1 - protein, 560 aa, Corynebacterium diphtheriae
MSRKLFASILIGALLGIGAPPSAHAGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - DNA, 1683 bp, Corynebacterium diphtheriae
gtgagcagaaaactgtttgcgtcaatcttaataggggcgctactggggataggggccccaccttcagcccatgcaggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagagttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcgtcggattccataggcgttcttgggtaccagaaaacagtagatcacaccaaggttaattctaagctatcgctattttttgaaatcaaaagctga

SEQ ID NO: 3 - DNA, 21 bp, Artificial Sequence
  SiRNA
cuuacgcugaguacuucgatt

SEQ ID NO: 4 - DNA, 21 bp, Artificial Sequence
  SiRNA
ucgaaguacucagcguaagtt

SEQ ID NO: 5 - DNA, 19 bp, Chlorocebus sabaeus
  misc_feature (1)..(19): lamin A/C
ctggacttccagaagaaca

SEQ ID NO: 6 - DNA, 19 bp, Rattus norvegicus
ggtggtgacgatctgggct

(19) United States
(12) Patent Application Publication
(10) Pub. No.: US 2012/0027843 A1
(43) Pub. Date: Feb. 2, 2012

(54) HB-EGF BOUND PROTEIN COMPLEX

(76) Inventors: Tetsuo Minamino, Osaka (JP); Eisuke Mekada, Osaka (JP); Asai Tomohiro, Shizuoka (JP); Seiji Takashima, Osaka (JP); Yoshihiro Asano, Osaka (JP)

(21) Appl. No.: 13/147,903

(22) PCT Filed: Feb. 3, 2010
(86) PCT No.: PCT/JP2010/051515

§ 371 Date: Oct. 18, 2011

(30) Foreign Application Priority Data
Feb. 4, 2009 (JP) 2009-023653

(51) Int. Cl. A61K 9/127; C07K 16/22; A61K 9/00; A61P 11/00; A61P 35/00; A61P 9/00; A61P 25/00; C07K 14/00; A61K 38/18

(52) U.S. Cl. 424/450; 530/350; 530/391.1; 424/400; 514/9.6

(57) ABSTRACT

A complex essentially comprising a protein having an action of binding to a membrane-bound HB-EGF to promote the uptake of the membrane-bound HB-EGF by cells, and a carrier.

### TECHNICAL FIELD

The present invention relates to an HB-EGF-bound protein complex capable of efficiently introducing an object substance, such as a drug and a gene, into cells; a medicament comprising the complex; a promoter for introduction of an object substance into cells; and a method for promoting introduction of an object substance into cells.

### BACKGROUND ART

In recent years, in the field of medicine, active development of a highly effective medicament which directly acts on an affected part or a target site with few side effects has been ongoing. In particular, the method called drug delivery system (DDS) attracts attention as a method for delivering an active ingredient of a drug etc. specifically to target cells and target tissue so that the active ingredient acts on an intended site. Also, in the field of recent molecular and cellular biology, gene introduction to target cells is indispensable in analysis of the structure, function, or control mechanism of a gene. Such a technology for introducing an object substance into cells is extremely important also in protein production, gene therapy, DNA vaccination, etc., which are important in the medical field.

As a method of introducing a substance, such as a drug, a physiologically active substance, and DNA, into cells, various methods are known. For example, in an experimental system of mammalian genes, a method with use of a viral vector (viral vector method), a method by lipofection with use of a liposome (liposome method), an ultrasonic method, and an electroporation method are performed as a method for introducing a gene etc. into cells.

As a viral vector, an adenovirus vector and a lentivirus vector are known, and transient inhibition of gene expression with use of an adenovirus vector having an inserted shRNA, and persistent gene expression inhibition with use of a lentivirus vector having an inserted shRNA are performed. However, the viral vector method has problems including immune responses induced by the viral vector, and modification of cellular genes by the viral vector. When used in a therapy etc., the method also involves ethical issues.

The liposome method is widely practiced because it is free from risks of immune responses and is highly safe unlike the viral vector method, does not damage cells unlike the ultrasonic method and the electroporation method, and does not need any special equipment. However, the liposome method has a problem of low efficiency of substance introduction into cells. This problem has prevented satisfactory introduction of an object substance into target cells in disease therapy or in an experimental system. In particular, it has been difficult to introduce a sufficient amount of an object substance, such as a drug and a gene, into cells, such as myocardial cells, nerve cells, and cancer cells.

Patent literature 1 discloses a method for introducing an object substance into cells by bringing the cells into contact with a composition which comprises a peptide having a specific amino acid sequence and the object substance. Patent literature 2 discloses a method for introducing an object substance into cells with use of a peptide having a specific amino acid sequence. However, these methods are not efficient enough to introduce an object substance specifically into myocardial cells etc., and there is still room for improvement.

Accordingly, development of safe technology to efficiently introduce an object substance specifically into cells, such as myocardial cells and nerve cells, has been desired.

### CITATION LIST

### Patent Literature

[Patent Literature 1]: JP-2004-534004 T
[Patent Literature 2]: Japanese Patent No. 3748561

### SUMMARY OF INVENTION

### Technical Problem

An object of the present invention is to provide an

HB-EGF-bound protein complex capable of efficiently introducing an object substance, such as a drug and a gene, into target cells; a kit comprising the complex; a therapeutic or prophylactic agent for cancer, cardiac failure, nerve disease, or pulmonary disease; a diagnostic agent; and a promoter and a promoting method for introduction of an object substance into cells.

### Solution to Problem

It is known that the membrane-bound type of a heparin-binding EGF-like growth factor (HB-EGF) is a diphtheria toxin receptor and that once the toxin binds to the receptor, the complex of the toxin and the HB-EGF is incorporated into cells (for example, Cell, 1992, 69, 1051-1061) through endocytosis. The present inventors made extensive research on a technology for introducing an object substance into target cells and found that anti-HB-EGF antibody modified liposomes (anti-HB-EGF antibody-bound liposomes) produced by modifying a PEG-liposome with an anti-HB-EGF antibody are efficiently introduced (taken) via a membrane-bound HB-EGF into cells. The introduction efficiency of the anti-HB-EGF antibody modified liposomes via membrane-bound HB-EGF into cells was about 60 times or higher than that of PEG-liposomes not modified with the anti-HB-EGF antibody. The inventors also found that cells which do not express HB-EGF hardly take anti-HB-EGF antibody modified liposomes and therefore use of the anti-HB-EGF antibody modified liposomes enables efficient introduction of an object substance into cells which express HB-EGF. Further, the inventors conceived an idea that, although it was previously difficult to introduce a substance into myocardial cells, nerve cells, or the like, use of the anti-HB-EGF antibody modified liposomes enables efficient introduction of a drug, a gene, or the like specifically into cells, such as failing myocardial cells, nerve cells, and pulmonary cells because these cells highly express HB-EGF (see, for example, Hypertens Res. 2008 February; 31 (2): 335-44 regarding the expression of HB-EGF in failing myocardial cells; and see, for example, Biochem Biophys Res Commun. 1993 Jan. 15; 190(1): 125-33 regarding the expression in the heart, lung, brain, or the like). In addition, the inventors found that it is possible to more efficiently introduce a drug or the like into cancer cells of ovarian cancer, uterine cancer, breast cancer, or the like because such cells highly express HB-EGF (see, for example, Cancer Sci. 2006 May; 97(5): 341-7 regarding the expression of HB-EGF in ovarian cancer; see, for example, Gynecol Oncol. 2007 January; 104 (1): 158-67 regarding the expression of HB-EGF in uterine cancer; and see, for example, Int J Surg Pathol. 2002 April; 10 (2): 91-9 regarding the expression of HB-EGF in breast cancer).

Based on the above findings, the inventors conducted further research and completed the present invention.

That is, the present invention relates to the following (1) to (10).

(1) A complex essentially comprising a protein having an action of binding to a membrane-bound HB-EGF to promote the uptake of the membrane-bound HB-EGF by cells, and a carrier.
(2) The complex according to the above (1), wherein the protein is an anti-HB-EGF antibody or a fragment thereof.
(3) The complex according to the above (1), wherein the protein is a diphtheria toxin mutant CRM197 or a fragment thereof.
(4) The complex according to any one of the above (1) to (3), wherein an object substance to be introduced into cells is enclosed in the carrier, or the object substance forms a complex together with the carrier.
(5) The complex according to the above (4), wherein the object substance is a drug for cancer, cardiac failure, nerve disease, or pulmonary disease, or a nucleic acid.
(6) A therapeutic or prophylactic agent for cancer, cardiac failure, nerve cell disease, or pulmonary disease, in which HB-EGF is highly expressed, the agent comprising a complex essentially comprising a protein having an action of binding to a membrane-bound HB-EGF to promote the uptake of the membrane-bound HB-EGF by cells, and a carrier.
(7) A diagnostic agent for cancer, cardiac failure, nerve cell disease, or pulmonary disease, in which HB-EGF is highly expressed, the agent comprising a complex essentially comprising a protein having an action of binding to a membrane-bound HB-EGF to promote the uptake of the membrane-bound HB-EGF by cells, and a carrier.
(8) A promoter for introduction of an object substance into cells, the agent comprising a complex essentially comprising a protein having an action of binding to a membrane-bound HB-EGF to promote the uptake of the membrane-bound HB-EGF by cells, and a carrier.
(9) A method for promoting introduction of an object substance into cells, the method comprising a step of adding a complex essentially comprising a protein having an action of binding to a membrane-bound HB-EGF to promote the uptake of the membrane-bound HB-EGF by cells, and a carrier; and an object substance to be introduced into cells, to cells expressing HB-EGF or a step of administering the complex and the object substance to an animal other than a human.
(10) A kit comprising a complex essentially comprising a protein having an action of binding to a membrane-bound HB-EGF to promote the uptake of the membrane-bound HB-EGF by cells, and a carrier.

The present invention also relates to

a therapeutic or prophylactic method for cancer, cardiac failure, nerve disease, or pulmonary disease, comprising a step of administering (A) a complex essentially comprising a protein having an action of binding to a membrane-bound HB-EGF to promote the uptake of the membrane-bound HB-EGF by cells, and a carrier, and (B) a drug for cancer, cardiac failure, nerve disease, or pulmonary disease, to a patient with cancer, cardiac failure, nerve disease, or pulmonary disease;
a diagnostic method for cancer, cardiac failure, nerve disease, or pulmonary disease, comprising a step of administering (A) a complex essentially comprising a protein having an action of binding to a membrane-bound HB-EGF to promote the uptake of the membrane-bound HB-EGF by cells, and a carrier, and (C) a diagnostic reagent, to a mammal including a human; and
a complex essentially comprising a protein having an action of binding to a membrane-bound HB-EGF to promote the uptake of the membrane-bound HB-EGF by cells, and a carrier, for use in therapy, prophylaxis, or diagnosis of cancer, cardiac failure, nerve disease, or pulmonary disease.

The present invention further includes a method for promoting introduction of an object substance into cells, the method comprising a step of administering a complex essentially comprising a protein having an action of binding to a membrane-bound HB-EGF to promote the uptake of the membrane-bound HB-EGF by cells, and a carrier; and an object substance to be introduced into cells to an animal including a human.

### Advantageous Effects of Invention

The present invention enables efficient introduction of a substance, such as a drug and a gene, into target cells, such as myocardial cells, and cancer cells, although such introduction was previously difficult. As a result, the present invention enables development of a novel therapeutic or diagnostic agent, etc., analysis of the structure, function, etc. of a gene, and the like.

### BRIEF DESCRIPTION OF DRAWINGS

FIG. 1 (a) shows the results of the expression of HB-EGF in Vero cells and Vero-H cells analyzed by Western blotting.

FIG. 1 (b) shows the results of the expression of β-actin, which is a control, analyzed by Western blotting.

FIG. 2 shows the results of SDS-PAGE of anti-HB-EGF monoclonal antibodies (IgG) digested with pepsin under reducing conditions.

FIG. 3 shows the binding (adhesion) of the anti-HB-EGF antibody-bound liposomes to (a) Vero cells and (b) Vero-H cells at 4° C.

FIG. 4 shows the uptake of the anti-HB-EGF antibody-bound liposomes by (a) Vero cells and (b) Vero-H cells at 37° C.

FIG. 5 shows the results of the expression of (a) human HB-EGF mRNA and (b) mouse HB-EGF mRNA in cultured rat myocardial cells.

FIG. 6 shows the results of the expression of endogenous genes in cultured rat myocardial cells.

FIG. 7 shows the results of the expression of (a) human HB-EGF protein and (b) mouse HB-EGF protein in cultured rat myocardial cells analyzed by Western blotting.

FIG. 8 shows the results of the localization of human HB-EGF protein in cultured rat myocardial cells analyzed by immunostaining with an anti-human HB-EGF antibody.

FIG. 9 shows the results of the localized expression of human HB-EGF protein ((a) and (c)) and of mouse HB-EGF protein ((b) and (d)) in cultured rat myocardial fibroblasts analyzed by immunostaining with an anti-human HB-EGF antibody.

FIG. 10 shows the results of the binding (adhesion) and the introduction of liposomes to/into cultured rat myocardial cells, or cultured rat myocardial cells where human HB-EGF is forcibly expressed.

FIG. 11 shows the results of the uptake of the anti-HB-EGF antibody-bound liposomes and control liposomes by a human breast cancer cell line (MDA-MB-231 cells).

FIG. 12 shows the experimental results of luciferase gene knockdown in Vero-H cells by the anti-HB-EGF antibody-bound liposomes containing siRNA ((a) 5 pmol or (b) 50 pmol) and by control liposomes.

FIG. 13 shows the experimental results of lamin gene knockdown in Vero-H cells by the anti-HB-EGF antibody-bound liposomes containing siRNA and by control liposomes.

FIG. 14 shows the inhibitory effect of the anti-HB-EGF antibody-bound liposomes containing doxorubicin on tumor growth in mice to which a human breast cancer cell line (MDA-MB-231 cells) was transplanted.

### DESCRIPTION OF EMBODIMENTS

Herein, “the complex or the promoter of the present invention promotes the introduction (uptake) of an object substance into cells” means that the introduction (uptake) of an object substance into cells proceeds more efficiently in an atmosphere of the complex or the promoter of the present invention as compared to the introduction (uptake) in the absence of the complex or the promoter.

Herein, “therapy” means not only to cure pathological conditions but also, to control progress and/or aggravation of symptoms to retard progression of pathological conditions, or to improve all or part of pathological conditions toward a cure and “prophylaxis” means to prevent, control, or delay the onset of pathological conditions.

The complex of the present invention is a complex essentially comprising a protein having an action of binding to a membrane-bound HB-EGF to promote the uptake of the membrane-bound HB-EGF by cells (hereinafter also referred to as “HB-EGF-bound protein”), and a carrier. Hereinafter, such a complex of the present invention is also referred to as simply “HB-EGF-bound protein complex”.

The HB-EGF-bound protein complex of the present invention binds to a membrane-bound HB-EGF (proHB-EGF) on a cell membrane by virtue of the HB-EGF-bound protein. This binding alters signaling and causes endocytosis, through which a complex composed of the complex of the present invention and a membrane-bound HB-EGF is incorporated into a cell. By this action, an object substance can be efficiently introduced into cells. The introduced object substance, such as a nucleic acid and a drug, will exert its action and work on proteins etc. in the cells or control the expression of other genes.

The complex of the present invention may be any complex as long as the complex essentially comprises an HB-EGF-bound protein and a carrier. Examples of the complex include a complex formed by binding, adhesion, or adsorption between an HB-EGF-bound protein and a carrier. Preferred is a complex formed by binding between an HB-EGF-bound protein and a carrier. The site of binding, adhesion, or adsorption between an HB-EGF-bound protein and a carrier is preferably different from the binding site between the HB-EGF-bound protein and a membrane-bound HB-EGF. In such a complex, the HB-EGF-bound protein may bind, adhere, or adsorb to the carrier directly or via a spacer etc.

The HB-EGF-bound protein of the present invention is not particularly limited as long as the protein is capable of recognizing a membrane-bound HB-EGF, binding to the membrane-bound HB-EGF, and causing endocytosis, but an anti-HB-EGF antibody or a fragment thereof is preferred. The antibody may be a monoclonal antibody or a polyclonal antibody, but a monoclonal antibody is preferred because of its high specificity for a membrane-bound HB-EGF.

The anti-HB-EGF antibody or a fragment thereof is preferably derived from an animal of the same species as the animal from which the membrane-bound HB-EGF expressing in the cells to which an object substance is introduced into derives. For example, in cases where an object substance is introduced into cells expressing human HB-EGF on the cell membrane, an anti-human HB-EGF antibody or a fragment thereof is preferred. The anti-HB-EGF antibody is preferably an anti-HB-EGF antibody to a membrane-bound HB-EGF present on the cell membrane of a mammal, such as a human, an ape, a cow, a sheep, a goat, a horse, a pig, a rabbit, a dog, a cat, a rat, a mouse, a guinea pig, and a human, and inter alia, an anti-human HB-EGF antibody is more preferred.

Preferred examples of the fragment of the anti-HB-EGF antibody of the present invention include a Fab fragment, a Fab′ fragment, and a F(ab′)₂ fragment obtainable by deleting the Fc region from the above-mentioned anti-HB-EGF antibody, and a scFv antibody obtainable by combining only the variable domains of the Fab region. Inter alia, a Fab′ fragment is preferred.

Preferred examples of the HB-EGF-bound protein in the present invention also include diphtheria toxin mutant CRM197, a fragment thereof, and the like. CRM197 is a nontoxic mutant of diphtheria toxin. This mutant has a mutation in its fragment A, but has the same binding activity to a membrane-bound HB-EGF as that of the wild type toxin. CRM197 is known to have a high affinity for the membrane-bound HB-EGF of mammals including particularly a human and an ape and excluding a mouse and a rat. Therefore, using a complex essentially comprising CRM197 and a carrier, it is possible to very efficiently introduce an object substance to cells expressing HB-EGF of a mammal that is not a mouse or a rat, but is particularly a human or an ape. CRM197 is described in J Biol. Chem. 1985 Oct. 5; 260 (22): 12148-53 etc. The amino acid sequence etc. of CRM197 are described in Japanese Patent No. 4203742. SEQ ID NO: 1 shows the amino acid sequence of CRM197. In the amino acid sequence of SEQ ID NO: 1, the sequence of the first 25 amino acids is a signal sequence. The domain consisting of the 378th to 535th amino acids of CRM197, the domain not comprising the signal sequence, is important for binding to a membrane-bound HB-EGF. As the fragment of CRM197, a fragment comprising the 378th to 535th amino acid sequence is preferred.

The HB-EGF-bound protein of the present invention may be labeled with a radioisotope, a fluorescent substance, a dye, or the like as long as the effect of the present invention is achieved.

The “carrier” in the present invention means a material capable of carrying an object substance described later, such as a nucleic acid and a drug, to target cells or a target site.

The carrier in the present invention preferably comprises at least one selected from the group consisting of a macromolecule, a fine aggregate, a fine particle, a microsphere, a nanosphere, a liposome, and an emulsion. Inter alia, a liposome is preferred. The liposome may be a multilamellar vesicle (MLV) or a unilamellar vesicle, such as a small unilamellar vesicle (SUV), a large unilamellar vesicle (LUV), and a giant unilamellar vesicle (GUV) as long as the liposome is a closed vesicle having a lipid bilayer structure. The size of the liposome is not particularly limited, but usually is about 50 to 3000 nm, preferably about 50 to 400 nm, and more preferably about 80 to 200 nm in diameter. The lipid constituting the liposome is not particularly limited, and any lipid that is usually used may be used. The liposome may be modified with polyethylene glycol (PEG) etc.

The complex of the present invention may further comprises an object substance to be introduced into cells (hereinafter, also referred to as simply an object substance). In cases where the complex of the present invention comprises an object substance, it is preferred that, for example, the object substance to be introduced into cells is enclosed in a carrier, or the object substance and a carrier form a complex.

The object substance to be introduced into cells can be suitably selected depending on the purpose, such as diagnosis, therapy, and research, and may be, for example, a nucleic acid, a drug, a peptide, a protein, a sugar, a complex thereof, a reagent, an investigational drug, or the like.

The nucleic acid may be a single strand or a double strand, and linear or annular. Examples of the nucleic acid include genomic DNA, cDNA, mRNA, antisense RNA, ribozyme, siRNA, short hairpin RNA (shRNA), and microRNA (miRNA). Examples of the nucleic acid include, in addition to DNA and RNA, analogs or derivatives thereof (for example, a peptide nucleic acid (PNA), and phosphorothioate DNA, etc.).

The drug is preferably a drug for cancer, cardiac failure, nerve disease, or pulmonary disease.

The drug for cancer is preferably a therapeutic or prophylactic drug for a cancer in which HB-EGF is highly expressed. Examples of the cancer in which HB-EGF is highly expressed include uterine cancer, ovarian cancer, and breast cancer. Examples of the drug for uterine cancer include cisplatin and doxorubicin, and examples of the drug for ovarian cancer include paclitaxel and doxyl. Examples of the drug for cardiac failure include a nonselective PDE inhibitor, such as a xanthine derivative; a PDEIII inhibitor, such as amrinone, milrinone, olprinone, pimobendan, and vesnarinone; a digitalis preparation; and others, such as an ACE inhibitor, an ATP preparation, nitrate drug, dipyridamole, nicorandil, a class III anti-arrhythmic drug, and bepridil (Bepricor). These drugs may also comprise a nucleic acid. For example, the drug for cardiac failure may comprise a nucleic acid, such as siRNA, shRNA, and miRNA, for a specifically expressed gene in failing myocardial cells.

Examples of the reagent include a fluorescent reagent, a pigment, and a diagnostic reagent.

The HB-EGF-bound protein complex of the present invention is a complex for efficient introduction of an object substance into cells highly expressing HB-EGF, for example, uterine cancer cells, ovarian cancer cells, breast cancer cells, etc. in which HB-EGF is highly expressed, failing myocardial cells, nerve cells, pulmonary cells, or the like. Therefore, the object substance is preferably a drug for cancer, cardiac failure, nerve disease, or pulmonary disease, or a nucleic acid, and more preferably a drug for cancer or cardiac failure, or a nucleic acid. In addition, a diagnostic reagent for a disease, such as cancer, cardiac failure, nerve disease, and pulmonary disease is also preferred.

The method for producing the complex of the present invention is not particularly limited, and may be a method comprising forming a complex of an HB-EGF-bound protein and a carrier by binding, adhesion, or adsorption by a known method. For example, in cases where the carrier is a liposome, the complex may be produced by, for example, binding the liposome and an HB-EGF-bound protein via a maleimide group. If desired, an object substance to be introduced into cells may be enclosed in or bound to the carrier beforehand. Alternatively, after the complex of an HB-EGF-bound protein and a carrier is formed by binding, adhesion, or adsorption by a known method, an object substance may be enclosed in or bound to the carrier as needed. Such a method for enclosing or binding a protein in/to a carrier is publicly known, and described in, for example, Ed. Akiyoshi Kazunari and Tsujii Kaoru, “Liposome ouyou no shintenkai—Jinkou saibou no kaihatsu ni mukete—”, NTS Inc., Jun. 1, 2005.

The HB-EGF-bound protein used for producing the HB-EGF-bound protein complex of the present invention is, as described above, preferably an anti-HB-EGF antibody or a fragment thereof, or a diphtheria toxin mutant CRM197 or a fragment thereof.

The anti-HB-EGF antibody may be a monoclonal antibody or a polyclonal antibody, but a monoclonal antibody is preferred because of its high specificity for a membrane-bound HB-EGF.

Anti-HB-EGF antibodies are commercially available and such a commercial antibody product may be used in the present invention. Examples of the commercial anti-human HB-EGF antibody include the following monoclonal antibodies etc., all of which are sold by Cosmo Bio Co., Ltd.: anti HB-EGF (H-88) (Catalog Number: SC28908), anti HB-EGF (C-18) (Catalog Number: SC1413), anti HB-EGF (E-10) (Catalog Number: SC74526), anti HB-EGF (G-11) (Catalog Number: SC74441), anti HB-EGF (Z14) (Catalog Number: SC74077L, SC74077), anti HB-EGF (C-14) (Catalog Number: SC21593), and anti HB-EGF (N-17) (Catalog Number: SC21591) made by Santa Cruz Biotechnology, Inc.; anti HB-EGF (Catalog Number: MAB259, AF259NA, MAB2591) and anti HB-EGF, Human, (Poly) (Trade name) (Catalog Number: BAF259) made by R&D Systems Inc.; anti HB-EGF (Catalog Number: PC319L) made by Calbiochem-Novabiochem International; anti HB-EGF (Catalog Number: 71503, 71501) made by Cosmo Bio Co., Ltd.; and anti-heparin-binding Egf-like Growth Factor (Catalog Number: LSC36646500) made by Lifespan Biosciences Inc.

Examples of the commercial anti-mouse HB-EGF antibody include anti HB-EGF (M-18) (Catalog Number: SC1414) made by Santa Cruz Biotechnology, Inc.

The HB-EGF antibody can also be prepared by a known method, for example, according to a method described in Mine N, Iwamoto R, Mekada E. HB-EGF promotes epithelial cell migration in eyelid development. Development, 2005 October; 132 (19): 4317-26 (p. 4318, Production method using HB^{del/del} mouse). For example, an anti-human HB-EGF monoclonal antibody can be produced by using a human antigen as the antigen in the above mentioned reference by Mine et al. As the preferable anti-human HB-EGF antibody in the present invention, HB-EGF monoclonal antibodies, mab clone: #3E9, #3H4, and #4G10, produced from hybridomas stored in the Mekada Lab., Department of Cell Biology, Research Institute for Microbial Diseases, Osaka University (3-1, Yamadaoka, Suita, Osaka 565-0871 Japan) can also be used. For example, the HB-EGF mab clone: #3E9 can be prepared according to the preparation method for the clone 4D9 described in the above mentioned reference by Mine et al.

A fragment of an anti-HB-EGF antibody can be obtained by a known method, for example, a method comprising chemically or enzymatically cleaving an anti-HB-EGF antibody, a method comprising creating a binding site of the antibody by a genetic engineering procedure, or the like. As a fragment of an anti-HB-EGF antibody, the Fab fragment, the Fab′ fragment, the F(ab′)₂ fragment, and the scFv antibody, of the HB-EGF antibody mentioned above can be preferably used.

The diphtheria toxin mutant CRM197 used for production of the HB-EGF-bound protein complex of the present invention can be obtained by, for example, a method described in Uchida T. et al., Diphtheria toxin and related proteins. I. Isolation and properties of mutant proteins serologically related to diphtheria toxin. J Biol Chem 1973; 248: 3838-44. Alternatively, the diphtheria toxin mutant can be obtained according to a method described in Japanese Patent No. 4203742, by culturing a stock of C7 (β197) lysogen (available as Corynebacterium diphtheriae C7 (β197)M1, which is lysogenized by C7 ((3197) phage, from American Type Culture Collection (ATCC) Bacteria Collection (No. 39255)) and then purifying CRM197 from the culture. Alternatively, the diphtheria toxin mutant can be synthesized, based on the amino acid sequence of CRM197 (SEQ ID NO: 1) in Japanese Patent No. 4203742, according to the peptide synthesis method described in, for example, Peptide Synthesis, Interscience, New York, 1966; The Proteins, Vol. 2, Academic Press Inc., New York, 1976; Peptide Gousei, Maruzen Co., Ltd., 1975; Peptide Gousei no Kiso to Jikken, Maruzen Co., Ltd., 1985; Zoku Iyakuhin no Kaihatsu, Vol. 14 Peptide Gousei, HirokawaShoten, 1991. Further, the diphtheria toxin mutant can be prepared by integrating the DNA sequence (SEQ ID NO: 2) which encodes CRM197 in a suitable expression vector, and introducing the vector into Escherichia coli etc.

A fragment of CRM197 can be obtained by a known method, for example, a method comprising chemically or enzymatically cleaving CRM197, a method of creating a peptide containing a binding site of CRM197 by a genetic engineering procedure, or the like. Alternatively, the fragment can be synthesized, based on the amino acid sequence of CRM197 (SEQ ID NO: 1) in Japanese Patent No. 4203742, by a peptide synthesis method.

The method for producing the carrier may be a publicly known method. Such a method is described in, for example, Ed. Akiyoshi Kazunari and Tsujii Kaoru, “Liposome ouyou no shintenkai—Jinkou saibou no kaihatsu ni mukete—”, NTS Inc., Jun. 1, 2005. By the Bangham (simplified hydration) method, reverse phase evaporation method, supercritical carbon dioxide method, etc., lipid hydration is performed to give a liposome solution.

The method for enclosing in a carrier an object substance to be introduced into cells, and the method for forming a complex of an object substance and a carrier are not particularly limited, and may be a publicly known method. For example, in cases where the carrier is a liposome and the object substance is soluble in water, the object substance can be enclosed in the aqueous phase inside the liposome by addition of the object substance to an aqueous solvent that is used for hydrating the lipid membrane at the time of liposome production. In cases where the object substance is soluble in oil, the object substance can be enclosed in the lipid bilayer of the liposome by addition of the object substance to an organic solvent that is used in liposome production.

The HB-EGF-bound protein complex of the present invention is efficiently incorporated into a cell via a membrane-bound HB-EGF, and therefore can be used to efficiently introduce an object substance described later into cells highly expressing HB-EGF. Since, for example, cancer cells such as uterine cancer cells, ovarian cancer cells and breast cancer cells, failing myocardial cells, nerve cells, pulmonary cells, or the like highly express HB-EGF, it is possible to efficiently introduce an object substance into such cells via an HB-EGF-bound protein complex.

For example, administration of a drug (a drug for cancer, cardiac failure, nerve disease, pulmonary disease, or the like) with the HB-EGF-bound protein complex of the present invention to an animal suffering from cancer accompanied by a high expression of HB-EGF such as uterine cancer, ovarian cancer, or breast cancer, cardiac failure, nerve disease, or pulmonary disease, enables efficient and specific introduction of the drug to cancer cells, failing myocardial cells, nerve cells, pulmonary cells, or the like. Therefore, such administration is effective in the therapy or prophylaxis of these diseases. In this case, the drug may be administered before, simultaneously with, or after the administration of the complex of the present invention. That is, the complex and the drug may be administered in either of the following two ways: (1) a composition comprising the complex of the present invention and the drug is prepared in order to administer the two at the same time (2) the complex of the present invention is administered to an animal so as to promote uptake by cells, such as cancer cells and failing myocardial cells, and then the drug is administered so as to be incorporated into the cells; or the drug is administered to the animal first, and then the complex of the present invention is administered. In the case of the above (1), the drug (a drug for cancer, cardiac failure, nerve disease, pulmonary disease, or the like) may be comprised in the complex of the present invention. Such a complex comprising a drug, such as a drug for cancer, cardiac failure, nerve disease, or pulmonary disease, as an object substance is a preferred embodiment of the present invention. Inter alia, it is preferred that the complex comprises a drug for cancer or cardiac failure.

A therapeutic or prophylactic agent for cancer, cardiac failure, nerve cell disease, or pulmonary disease in which HB-EGF is highly expressed, the agent comprising a complex essentially comprising an HB-EGF-bound protein and a carrier (HB-EGF-bound protein complex) is also one of the present invention. A preferred embodiment is a therapeutic or prophylactic agent for cancer or cardiac failure accompanied by an increased expression of HB-EGF. The therapeutic or prophylactic agent of the present invention may further comprise one or more drugs selected from the group consisting of a drug for cancer, a drug for cardiac failure, a drug for nerve disease, and a drug for pulmonary disease, and a pharmaceutically acceptable ingredient depending on the formulation. In the present invention, the HB-EGF-bound protein complex may comprise the above-mentioned drug for cancer, cardiac failure, nerve disease, or pulmonary disease. The formulation of the therapeutic or prophylactic agent of the present invention is preferably a formulation for parenteral administration, and examples thereof include an injection, an intravenous fluid, an ointment, a gel, a cream, a patch, a spray, a spray, etc. Inter alia, an injection is preferred.

The injection for parenteral administration may be an aqueous injection or an oily injection. To prepare an aqueous injection, according to a publicly known method, an aqueous solvent (water for injection, purified water, or the like), to which a pharmaceutically acceptable additive is appropriately added as needed, and an HB-EGF-bound protein complex (and a drug if desired) are mixed. The mixture is sterilized by filtration with a filter etc. and then charged into a sterile container. Examples of the pharmaceutically acceptable additive include isotonic agents, such as sodium chloride, potassium chloride, glycerol, mannitol, sorbitol, boric acid, borax, glucose, and propylene glycol; buffering agents, such as phosphate buffer, acetic acid buffer, boric acid buffer, carbonic acid buffer, citric acid buffer, tris buffer, glutamic acid buffer, and epsilon aminocaproic acid buffer; preservatives, such as methyl parahydroxybenzoate, ethyl parahydroxybenzoate, propyl parahydroxybenzoate, butyl parahydroxybenzoate, chlorobutanol, benzyl alcohol, benzalkonium chloride, sodium dehydroacetate, disodium edetate, boric acid, and borax; thickeners, such as hydroxyethyl cellulose, hydroxypropyl cellulose, polyvinyl alcohol, and polyethylene glycol; stabilizers, such as sodium hydrogensulfite, sodium thiosulfate, disodium edetate, sodium citrate, ascorbic acid, and dibutyl hydroxytoluene; and pH adjusters, such as hydrochloric acid, sodium hydroxide, phosphoric acid, and acetic acid.

In addition, the injection may also contain an appropriate solubilizing agent, for example, an alcohol, such as ethanol; a polyalcohol, such as propylene glycol and polyethylene glycol; a nonionic surfactant, such as polysorbate 80, polyoxyethylene hydrogenated castor oil 50, lysolecithin, and Pluronic polyol; and the like. Further, the injection may contain a protein, such as bovine serum albumin and keyhole limpet hemocyanin; a polysaccharide, such as aminodextran; and the like. To prepare an oily injection, sesame oil, soy bean oil, or the like may be used as an oily solvent. In this case, benzyl benzoate, benzyl alcohol or the like may be blended as a solubilizing agent. The prepared injection solution is usually charged into a suitable ampule, vial, or the like. Liquid preparations, such as an injection preparation may be stored after water removal by cryopreservation, lyophilization, or the like. To the lyophilized formulation, distilled water for injection or the like is added at the time of use to dissolve the formulation.

The amount of the HB-EGF-bound protein complex contained in the therapeutic or prophylactic agent of the present invention varies depending on the formulation and the route of administration of the therapeutic or prophylactic agent, but usually may be suitably selected from the range of about 0.0001 to 100% relative to the final formulation.

The route of administration of the therapeutic or prophylactic agent of the present invention to a mammal including a human may be parenteral administration, such as intravenous, intraperitoneal, hypodermic, and intranasal. Depending on the cells or the site, the agent may be directly administered locally. For example, in cases where the drug or the like is introduced into myocardial cells, the therapeutic or prophylactic agent comprising an HB-EGF-bound protein complex can be directly administered into myocardial cells by intramyocardial injection, the CARTO (registered trademark) system (by Johnson & Johnson, Medical Company), etc. Alternatively, the therapeutic or prophylactic agent can be intracoronarily administered after treatment for increasing vascular permeability.

When the therapeutic or prophylactic agent of the present invention is administered, the above-mentioned drug, such as a drug for cancer, cardiac failure, nerve disease, or pulmonary disease, may be administered before, simultaneously with, or after the administration of the therapeutic or prophylactic agent depending on the disease. For example, the therapeutic or prophylactic agent and the drug may be administered in either of the following two ways: (1) the therapeutic or prophylactic agent comprising the HB-EGF-bound protein and the drug (a drug for cancer, cardiac failure, nerve disease, pulmonary disease, or the like) is prepared and then administered (2) the therapeutic or prophylactic agent is administered to an animal so as to promote uptake by cells, such as cancer cells and failing myocardial cells, and then the drug is administered so as to be incorporated into the cells; or the drug is administered to the animal first, and then the therapeutic or prophylactic agent is administered. In the case of the above (1), the drug (a drug for cancer, cardiac failure, nerve disease, pulmonary disease, or the like) may be comprised in the HB-EGF-bound protein complex. In cases where the therapeutic or prophylactic agent of the present invention comprises the drug, the amount of the drug can be suitably adjusted depending on the type etc. of the drug. In cases where the therapeutic or prophylactic agent of the present invention and the drug are administered separately, the amount of the drug also can be suitably adjusted depending on the type etc. of the drug.

The dosage and the frequency of administration of the therapeutic or prophylactic agent of the present invention can be suitably adjusted depending on the type and amount of the drug administered with the HB-EGF-bound protein complex (a drug for cancer, cardiac failure, nerve disease, pulmonary disease, or the like), the body weight of the patient, the disease, etc. The dosage etc. is preferably adjusted so that the drug is efficiently introduced into the affected part or target cells.

The subject to whom the therapeutic or prophylactic agent of the present invention is administered, is preferably a mammal suffering from one or more diseases selected from the group consisting of cancers accompanied by a high expression of HB-EGF such as uterine cancer, ovarian cancer, and breast cancer, cardiac failure, nerve disease, and pulmonary disease. When the agent is used for therapy, the subject is more preferably a mammal suffering from cancer in which HB-EGF is highly expressed or cardiac failure. When the agent is used for prophylaxis, the subject is preferably a mammal having the potential of developing one or more diseases selected from the group consisting of cancers accompanied by a high expression of HB-EGF such as uterine cancer, ovarian cancer, and breast cancer, cardiac failure, nerve disease, and pulmonary disease. The mammal is preferably a human, an ape, a cow, a sheep, a goat, a horse, a pig, a rabbit, a dog, a cat, a rat, a mouse, a guinea pig, or the like, and more preferably a human suffering from or having the potential of developing such a disease.

The above-mentioned HB-EGF-bound protein complex can also be used for diagnosis or detection of cancer, cardiac failure, etc. in which HB-EGF is highly expressed. A diagnostic agent for cancer, cardiac failure, nerve cell disease, or pulmonary disease accompanied by a high expression of HB-EGF, the agent comprising the HB-EGF-bound protein complex is also one of the present invention. For example, administration of a fluorescent reagent, a diagnostic reagent, etc. before, simultaneously with, or after the administration of the HB-EGF-bound protein complex of the present invention to an animal suspected to have developed uterine cancer, ovarian cancer, or breast cancer, in which HB-EGF is highly expressed, cardiac failure, nerve cell disease, or pulmonary disease, enables efficient introduction of such a reagent into cancer cells, failing myocardial cells, nerve cells, pulmonary cells, or the like. By subsequent detection of the reagent according to a publicly known method, detection of an affected part, diagnosis of a disease, etc. can be performed. The diagnostic reagent etc. may be comprised in the HB-EGF-bound protein complex. The formulation and the route of administration of the diagnostic agent of the present invention are the same as those of the above-mentioned therapeutic or prophylactic agent, and can be suitably selected. The type, the route of administration, the dosage, etc. of the diagnostic reagent used with the HB-EGF-bound protein complex can be suitably selected depending on the type of the disease etc.

By adding the above-mentioned HB-EGF-bound protein complex to cells (target cells) in vitro, or to an animal in vivo, introduction of the object substance into cells can be promoted. Such a method for promoting introduction of an object substance into cells, the method comprising a step of adding the HB-EGF-bound protein complex and an object substance to be introduced into cells, to cells or a step of administering the same two to an animal is also one of the present invention.

In the method of the present invention for promoting introduction into cells (hereinafter referred to as the promotion method of the present invention), the target cell is preferably a cell highly expressing HB-EGF, and particularly preferably a cell highly expressing HB-EGF on the membrane. In cases where the target cell is a cell not expressing HB-EGF or a cell lowly expressing HB-EGF, high expression of HB-EGF can be achieved by introducing a gene that expresses HB-EGF into the cell by a publicly known method, for example, a method with use of adenovirus vector as described in the Examples, a method comprising introducing a vector obtained by integrating a gene that expresses HB-EGF into a suitable plasmid vector by lipofection, a calcium phosphate method, or an electroporation method, or the like. Examples of the target cell include cells derived from a mammal, such as a human, an ape, a cow, a sheep, a goat, a horse, a pig, a rabbit, a dog, a cat, a rat, a mouse, and a guinea pig. Nondividing cells, such as a myocardial cell and a nerve cell, are also preferred.

Examples of the animal to which the HB-EGF-bound protein complex is administered in the promotion method of the present invention include the above-mentioned mammals. According to the promotion method of the present invention, an object substance, such as an investigational drug, a drug, a reagent, and a gene, can be efficiently introduced into cancer cells, failing myocardial cells, nerve cells, pulmonary cells, or the like in an affected part of an animal suffering from, for example, cancer accompanied by a high expression of HB-EGF such as uterine cancer, ovarian cancer, or breast cancer, cardiac failure, nerve disease, or pulmonary disease.

In the method of the present invention, a composition comprising an object substance and the HB-EGF-bound protein complex may be prepared for addition to target cells or administration to an animal. Alternatively, the HB-EGF-bound protein complex may be added to target cells or administered to an animal so as to promote uptake by cells before the object substance is added so as to be incorporated into the cells. Alternatively, the object substance may be added to the target cells or administered to the animal before the addition or administration of the HB-EGF-bound protein complex. The object substance may be comprised in the HB-EGF-bound protein complex. In this case, it is preferred that the object substance is enclosed in a carrier, or the object substance and a carrier form a complex. The amounts of the HB-EGF-bound protein complex and the object substance to be added or administered may be suitably selected depending on the type of the cells or the animal, the object substance, or the like.

In the promotion method of the present invention, it is preferred that a composition comprising the HB-EGF-bound protein complex is added to cells or administered to an animal. Such a composition comprising the HB-EGF-bound protein complex is useful as a promoter for promoting introduction of an object substance to cells. Such a promoter comprising the HB-EGF-bound protein complex is also one of the present invention. The promoter of the present invention for promoting introduction into cells may further comprise an object substance, and the object substance may be comprised in the HB-EGF-bound protein complex.

The formulation of the promoter of the present invention is not particularly limited, and in cases where the promoter is administered to an animal, the formulation may be the same as that of the above-mentioned therapeutic or prophylactic agent and preferably an injection or a intravenous fluid. The promoter may further comprise a pharmaceutically acceptable ingredient depending on the formulation. The route of administration, the dosage, etc. of the promoter of the present invention are also the same as those of the therapeutic or prophylactic agent mentioned above.

Examples of the formulation for in vitro addition to cells include a dispersion liquid of the HB-EGF-bound protein complex. Examples of the dispersing solvent include buffer solutions, such as a physiological saline, a phosphate buffer solution, a citric acid buffer solution, and an acetic acid buffer solution. Additives, such as saccharides, a polyhydric alcohol, a water soluble polymer, a nonionic surfactant, an anti-oxidant, a pH regulator, and a hydration accelerator, may be added to the dispersion liquid.

Another example of the formulation for in vitro addition to cells may be a dried matter (for example, a lyophilized matter, a spray-dried matter, etc.) of a dispersion liquid of the HB-EGF-bound protein complex. The dried matter can be used as a dispersion liquid of the HB-EGF-bound protein complex by addition of a buffer solution, such as a physiological saline, a phosphate buffer solution, a citric acid buffer solution, and an acetic acid buffer solution.

In cases where the promoter of the present invention is added to cells in vitro, the promoter is added to or brought into contact with cells cultured in a suitable culture medium, for example. Subsequently, a step of incubation of the cells and the promoter, a step of washing the cells, a step of collecting the cells, etc. may be performed. Introduction of an object substance into cells can be confirmed by a publicly known method after cell lysis, for example.

A kit comprising a complex essentially comprising an HB-EGF-bound protein and a carrier is also one of the present invention.

The kit of the present invention may suitably comprise an object substance, cells into which the object substance is introduced, a buffer solution, a culture medium, etc. The preferred embodiments of the HB-EGF-bound protein complex are as described above, and the HB-EGF-bound protein complex may comprise an object substance. The object substance is preferably an investigational drug, a nucleic acid, or a drug. The cells into which the object substance is introduced are not particularly limited, and preferably nondividing cells, such as myocardial cells and nerve cells. In cases where the cells into which the object substance is introduced are cells not expressing HB-EGF or cells lowly expressing HB-EGF, high expression of HB-EGF can be achieved by introducing a gene that expresses HB-EGF into the cell by a publicly known method, for example, a method with use of a virus vector of adenovirus etc. as described in the Examples, a method comprising introducing a vector obtained by integrating a gene that expresses HB-EGF into a suitable plasmid vector by lipofection, a calcium phosphate method, or an electroporation method, or the like.

The kit of the present invention can be suitably used for introduction of a nucleic acid, an investigational drug, etc. to cultured myocardial cells or cultured nerve cells, which has been technically difficult.

### EXAMPLES

Hereinafter, the present invention will be described in more detail by reference to Examples, but is not limited thereto.

### Test Example 1

### (I) Preparation of Vero-H cells

Vero cells (HB-EGF low-expressing cells) are available from American Type Culture Collection (ATCC). Vero-H cells (HB-EGF high-expressing cells), which highly express human HB-EGF, were prepared according to the method described in Goishi K., et al. Phorbol ester induces the rapid processing of cell surface heparin-binding EGF-like growth factor: conversion from juxtacrine to paracrine growth factor activity. Mol Biol Cell 1995; 6: 967-980.

The Vero cells (HB-EGF low-expressing cells) were maintained in a MEM culture medium containing non-essential amino acids (solution containing 10% fetal bovine serum (FBS), 100 units/mL penicillin, and 100 μg/mL streptomycin) in an atmosphere of 5% carbon dioxide at 37° C. The Vero-H cells (HB-EGF high-expressing cells) were maintained in a MEM culture medium containing non-essential amino acids (solution containing 10% fetal bovine serum (FBS), 100 units/mL penicillin, 100 μg/mL streptomycin, and 1 μg/mL geneticin) in an atmosphere of 5% carbon dioxide at 37° C. When the cells reached confluence, a 0.25% solution of trypsin in EDTA-PBS (−) was added to strip the cell.

Western blotting was performed to confirm that the Vero-H cells highly express HB-EGF.

### (II) Western Blotting

### (1) Cell Seeding

The above Vero cells or Vero-H cells were seeded into wells of a 24-well plate (3.0×10⁴ cells/well) and incubated at 37° C. for 48 hours.

### (2) Sample Preparation

The cells cultured in (1) were washed with ice-cooled PBS(−) three times. Subsequently, the cells were lysed using 50 mM Tris-HCl (pH 7.4), 1% Triton-X, and 0.15 M NaCl (containing protein inhibitor) (4° C., 1 hour). The cell lysate was centrifuged (at 20,000 at 4° C. for 10 minutes), and the supernatant was used as a sample. The sample was measured for the absorbance at 280 nm to determine the protein amount therein, and then subjected to Western blotting.

### (3) Western Blotting

With 2.35 μL of 4× Sample Buffer ME(+), 7.04 μL of the sample was diluted. Then, this diluted solution was boiled at 95° C. for 5 minutes. The sample (20 μg) and a marker were applied to a gel for Western blotting. As the marker, Pre-stained marker (5 μL, made by Bio-Rad) and Magicmark (5 μL, made by Invitrogen) were used. Electrophoresis was performed at 5 mA (stacking gel) and at 10 mA (running gel). The stacking gel was separated from the running gel, and proteins were transferred to a nitrocellulose filter at 40 V over 90 minutes (blotting). Next, the filter was shaken in a blocking solution (TTBS containing 5% (w/v) BSA) at room temperature for 1 hour for blocking. TTBS is a TBS (tris buffered saline) containing 0.05% Tween 20.

The blocked nitrocellulose filter was soaked in a TTBS (containing 5% (w/v) BSA) containing a primary antibody, incubated with shaking at 4° C. overnight, and then washed with TTBS (5 minutes×3 times). As the primary antibody to HB-EGF, 0.1 μg/mL of an anti-HB-EGF antibody (made by R&D Systems, Inc. diluted 2500-fold with TTBS containing 5% (w/v) BSA) was used. Next, the filter was soaked in a TTBS (containing 5% (w/v) BSA) containing a secondary antibody, shaken at room temperature for 1 hour, and then washed with TTBS (5 minutes×3 times). As the secondary antibody, an anti-HB-EGF antibody, anti-goat IgG (made by Santa Cruz Biotecnology, diluted 4000-fold with TTBS containing 5%. (w/v) BSA) was used. The location of the objective antigen was detected using the ECL system (made by Amersham).

β-actin was selected as a control. As the primary antibody to β-actin, an anti-actin rabbit IgG (diluted 4000-fold with TTBS containing 5% (w/v) BSA) was used. As the secondary antibody, an HRP-bound-anti-rabbit IgG (made by Amersham, diluted 5000-fold with TTBS containing 5% (w/v) BSA) was used.

### (4) Results

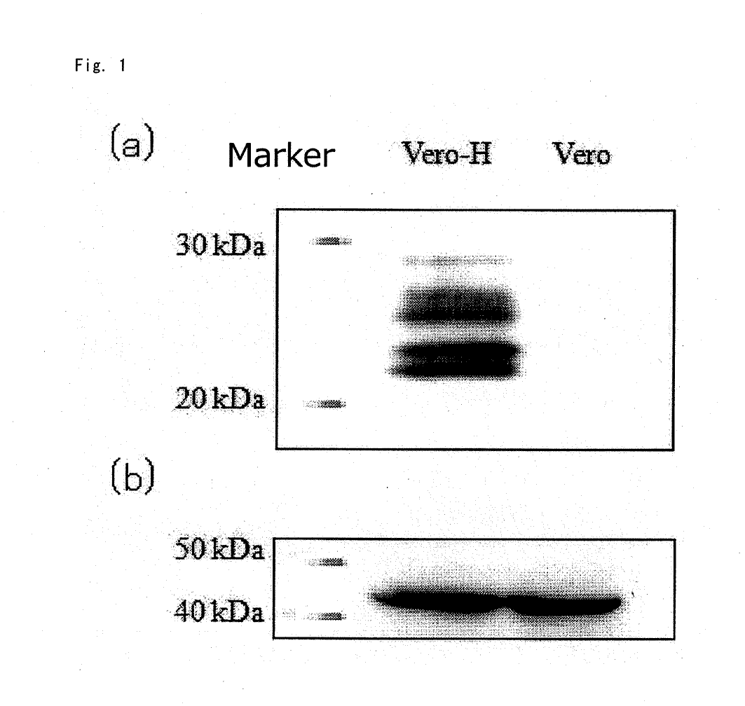

FIG. 1 (a) shows the results of the expression of HB-EGF in Vero cells and Vero-H cells analyzed by Western blotting. In FIGS. 1 (a) and (b), the left lane, the middle lane, and the right lane are of a marker, Vero-H cells, and Vero cells, respectively. As shown in FIG. 1 (a), in the case of Vero-H cells (middle lane), bands are observed at about 20 to 30 kDa, confirming high expression of HB-EGF.

FIG. 1 (b) shows the results of the expression of β-actin (control) in Vero cells and Vero-H cells analyzed by Western blotting. FIG. 1 (b) shows that β-actin equally is equally expressed in Vero cells and Vero-H cells.

### Example 1

### (I) Preparation of Anti-HB-EGF Antibody-Bound Peg Liposomes

(1) Preparation of F(ab′)₂ anti-HB-EGF antibody

Preparation of F(ab′)₂ anti-HB-EGF antibody was performed according to the conventional method.

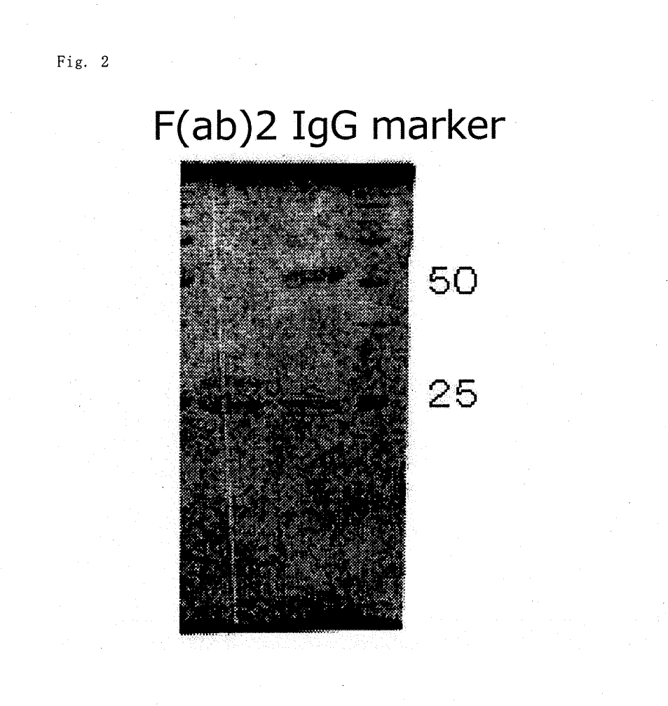

Human IgG (monoclonal antibody produced from a hybridoma stored in the Mekada Lab., Department of Cell Biology, Research Institute for Microbial Diseases, Osaka University (3-1, Yamadaoka, Suita, Osaka 565-0871 Japan) (HB-EGF mab clone: #3E9)) was digested with pepsin (37° C., 10 hours). The hybridoma and the monoclonal antibody, HB-EGF mab clone: #3E9, can be prepared, for example, using a human antigen as the antigen, according to a method described in Mine N, Iwamoto R, Mekada E. HB-EGF promotes epithelial cell migration in eyelid development. Develooment, 2005 October; 132 (19): 4317-26 (p. 4318, Production method using HB^{del/del} mouse) (The HB-EGF mab clone: #3E9 can be prepared according to the preparation method of clone 4D9 described in this reference). The F(ab′)₂ fragment was purified by gel filtration with the use of Ultrogel AcA54 (made by PALL Life Sciences). Using the absorbance at 280 nm as an index, eluate fractions containing the F(ab′)₂ fragment were collected. In order to check the cleavage and purification of the F(ab′)₂ fragment, SDS-polyacrylamide gel electrophoresis was performed. A band at about 30 kDa confirmed that F(ab′)₂ fragment was obtained (FIG. 2). In FIG. 2, since the SDS gel electrophoresis was carried out in reducing conditions, the band of the F(ab′)₂ fragment is not observed at 110 kDa. However, in the IgG lane, the band at about 50 kDa is missing, showing that preparation of the F(ab′)₂ fragment was completely achieved. This F(ab′)₂ fragment was used as an F(ab′)₂ anti-HB-EGF antibody.

### (2) Preparation of Fluorescently-Labeled Liposomes

By the thin film method, hydrogenated soybean phosphatidylcholine/cholesterol/polyethyleneglycol-bound hydrogenated soybean phosphatidylcholine/maleimidized polyethyleneglycol-bound hydrogenated soybean phosphatidylcholine (HSPC/Chol/DSPE-PEG-mal) liposomes were prepared.

Using a microsyringe, part of a lipid solution (hydrogenated soybean phosphatidylcholine/cholesterol/polyethyleneglycol-bound hydrogenated soybean phosphatidylcholine/maleimidized polyethyleneglycol-bound hydrogenated soybean phosphatidylcholine/octadecylindocarbocyanine (fluorescent substance)=1/0.67/0.03/0.003 (0 in the case of control liposome)/0.05 (molar ratio)) was placed into a flask. After the solvent was thoroughly distilled off, saline at 60° C. was added thereto, and the mixture was mixed with a vortex mixer for complete hydration. The liquid was passed 10 times through a polycarbonate membrane filter (with 100 nm pores) set in an extruder to adjust the particle diameter of liposomes.

### (3) Preparation of Fluorescently-Labeled Anti-HB-EGF Antibody-Bound Liposome

The F(ab′)₂ anti-HB-EGF antibody prepared above was mixed with cysteamine hydrochloride (cysteamine-HCl), and the mixture was incubated at 37° C. for 1.5 hours so that the F(ab′)₂ anti-HB-EGF antibody was reduced. Subsequently, the obtained Fab′ anti-HB-EGF antibody was isolated by gel filtration using Sepharose 4 FastFlow (made by GE healthcare Co.). To the liposome solution prepared in the above (2), the Fab′ anti-HB-EGF antibody in an equimolar amount to the maleimidized polyethyleneglycol-bound hydrogenated soybean phosphatidylcholine was added for reaction at 4° C. for 20 hours. After the reaction, 0.1 M N-ethylmaleimide in 10 times the molar amount of the Fab′ anti-HB-EGF antibody was added to block unreacted sulfhydryl groups. The fluorescently-labeled liposome to which the Fab′ anti-HB-EGF antibody was bound (fluorescently-labeled anti-HB-EGF antibody-bound liposome) was purified by gel filtration using a sepharose carrier. The fluorescently-labeled anti-HB-EGF antibody-bound liposome was detected by absorbance determination at 280 nm and 610 nm.

### (II) Cell Culture

The Vero cells (HB-EGF low-expressing cells) were maintained in a MEM culture medium containing non-essential amino acids (solution containing 10% fetal bovine serum (FBS), 100 units/mL penicillin, and 100 μg/mL streptomycin) in an atmosphere of 5% carbon dioxide at 37° C. The Vero-H cells (HB-EGF high-expressing cells) were maintained in a MEM culture medium containing non-essential amino acids (solution containing 10% fetal bovine serum (FBS), 100 units/mL penicillin, 100 μg/mL streptomycin and 1 μg/mL geneticin) in an atmosphere of 5% carbon dioxide at 37° C. When the cells reached confluence, a 0.25% solution of trypsin in EDTA-PBS(−) was added to strip the cell.

The obtained cells were used for uptake test.

### (III) Uptake Test

The above cultured Vero cells or Vero-H cells were seeded into wells of a 24-well plate (3.0×10⁴ cells/well) and incubated at 37° C. for 48 hours. Subsequently, the culture medium was removed from each well of the plate, a MEM culture medium containing non-essential amino acids containing the fluorescently-labeled anti-HB-EGF antibody-bound liposome (HSPC/Chol/DSPE/DSPE-PEG-mal/DiI) prepared in the above (1) (2) and (3), or the fluorescently-labeled liposome (HSPC/Chol/DSPE/DiI) as a control was added in an amount shown in Table 1. Table 1 shows the liposome concentration in each sample prepared.

TABLE 1

| | MEM culture | |
|---|---|---|
| | medium containing | Final concentration |
| Liposome | non-essential amino acids | of liposome |
| (μL) | (μL) | (mM) |
| 25 | 475 | 0.05 |
| 50 | 450 | 0.1 |
| 100 | 400 | 0.2 |

After incubating the culture medium containing the liposomes and the cells at 4° C. or 37° C. for 4 hours, the cells were washed 3 times with phosphate buffered saline. The washed cells were lysed by addition of a lysis buffer solution (tris-hydrochloric acid buffer solution containing 0.1% sodium dodecyl sulfate (SDS)). After this lysate was collected into a 1.5-mL eppendorf tube and then centrifuged at 1,000 g for 10 minutes, the supernatant was collected. The fluorescence intensity of the sample was determined with a fluorophotometer (Ex: 549 nm, Em: 592 nm). The fluorescence intensity of each sample was corrected for the protein amount.

Further, after the above fluorescently-labeled anti-HB-EGF antibody-bound PEG liposomes were added to the Vero cells or Vero-H cells, the intracellular localization of the liposomes was examined by confocal laser microscopy.

### (IV) Results

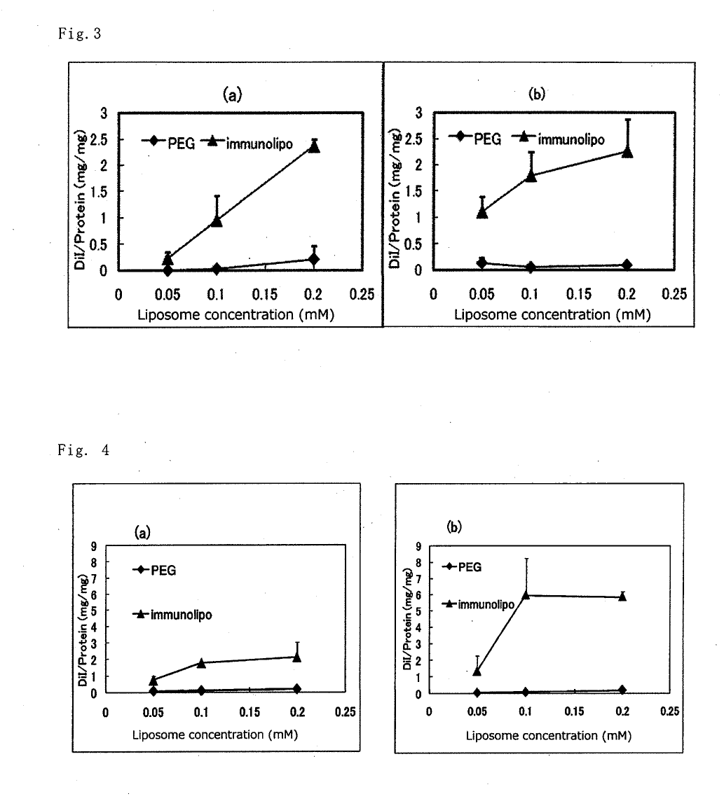

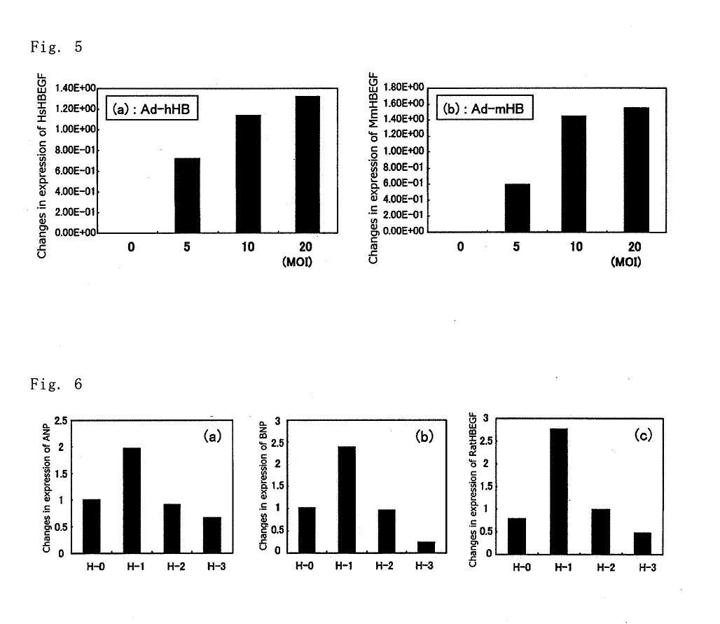

The results are shown in FIGS. 3 and 4. The vertical axis of the graph indicates the fluorescence intensity (Ex: 549 nm, Em: 592 nm). A greater value on the vertical axis means more binding (adhesion) of liposomes to cells and more intake of liposomes by cells. In the graph, the lozenge represents the control liposome and the triangle represents the Fab′ anti-HB-EGF antibody-bound liposome.

FIG. 3 shows the binding (adhesion) of the liposomes to cells at 4° C., and FIG. 4 shows the uptake of the liposomes by cells at 37° C. At 4° C., uptake of the liposome by cells hardly occurred. In both of the left graphs of FIGS. 3 and 4 (FIG. 3 (a) and FIG. 4 (a)), where the Vero cells (HB-EGF low-expressing cells) were used, the binding (adhesion) or uptake of the anti-HB-EGF antibody-bound liposomes by cells was only slightly more than that of the control liposomes. By contrast, in the right graphs (FIG. 3 (b) and FIG. 4 (b)), where the Vero-H cells (HB-EGF high-expressing cells) were used, the binding (adhesion) or uptake of the anti-HB-EGF antibody-bound liposomes by cells largely surpassed that of the control liposomes.

The above results suggest that enhanced uptake of the anti-HB-EGF antibody-bound liposomes (anti-HB-EGF antibody modified liposomes) by cells which highly express HB-EGF, such as failing myocardial cells, uterine cancer cells, ovarian cancer cells, and breast cancer cells, can be expected.

In addition, analysis by confocal laser microscopy revealed that the anti-HB-EGF antibody-bound PEG liposomes were positively taken into the Vero-H cells (HB-EGF high-expressing cell strain) and internalized therein.

Even when another anti-HB-EGF antibody (for example, a commercial anti-HB-EGF antibody), CRM197, or the like is used in the preparation of the anti-HB-EGF antibody-bound liposomes in Example 1 instead of the anti-HB-EGF antibody used above, the same results as in Example 1 can be obtained. Likewise, even when an anti-HB-EGF antibody that is different from the anti-HB-EGF antibody used above (for example, a commercial anti-HB-EGF antibody), CRM197, or the like is used for the anti-HB-EGF antibody-bound liposomes, the same results can be obtained in the Examples below.

### Example 2

Based on the experimental results obtained using the Vero cells and the Vero-H cells in Example 1, the following experiments were conducted for the purpose of confirming that the anti-HB-EGF antibody-bound liposomes which specifically recognize human HB-EGF are actually taken into myocardial cells.

Specifically, cultured myocardial cells from newborn rats were made to express human HB-EGF, and in this human HB-EGF expression system, uptake of the anti-HB-EGF antibody-bound liposome was examined.

### (I) Preparation of Adenovirus Construct

An adenovirus construct which expresses human HB-EGF (Ad-HsHBEGF) and an adenovirus construct which expresses mouse HB-EGF (Ad-MmHBEGF) were separately prepared. In addition, as an adenovirus control, an adenovirus construct which expresses LacZ was prepared (Ad-LacZ).

For the preparation of the adenovirus constructs (adenovirus vectors), Gateway system made by Invitrogen was used. Each construct (vector) was prepared by introduction of a human HB-EGF gene (NM_{—}001945.1) or a mouse HB-EGF gene (NM_{—}010415.1) into an entry vector (pENTR/D-TOPO) and subsequent conversion to an adenovirus expression vector (pAD/CMV/V5-DEST). Such vector preparation can be performed according to, for example, the method described in Hartley J L, Temple G F, Brasch M A. (2000) DNA cloning using in vitro site-specific recombination. Genome, Res, Vol. 10 (11): 1788-95.

(II) Examination of Expression in Cultured Myocardial Cells from Newborn Rats

Cultured myocardial cells from newborn rats, Wistar rat, (made by Kiwa Laboratory Animals Co., Ltd.) were seeded into wells of a 6-well plate (1.5×10⁶ cells/well). To this, one of the Ad-HsHBEGF, Ad-MmHBEGF, and Ad-LacZ prepared above was added and mixed to infect the cultured rat myocardial cells. The cultured rat myocardial cells were infected with Ad-HsHBEGF, Ad-MmHBEGF, or Ad-LacZ, at 0 to 20 of Multiplicity of Infection (MOI). When the number of cells is 1.5×10⁶, MOI10 is equivalent to 1.5×10⁻¹° PFU. When MOI was 10, 1.5 μL of a viral solution of 1.0×10⁻¹° PFU/μL was added to each well. After infection, Real-time PCR was performed to determine the mRNA expression level of human HB-EGF or mouse HB-EGF in the cultured rat myocardial cells. The results showed that the human HB-EGF gene and the mouse HB-EGF gene were expressed in the cultured rat myocardial cells in the mRNA level in an infection dosage-dependent manner (FIGS. 5 (a) and (b)). Further, whether the introduction of Ad-HsHBEGF or Ad-MmHBEGF has an influence on the expression of endogenous genes (mRNA of ANP, BNP, and rat HB-EGF) was investigated. The results showed that the expression of endogenous genes including the endogenous HB-EGF (rat HB-EGF) was not affected by the HB-EGF forcibly expressed by introduction of Ad-HsHBEGF or Ad-MmHBEGF (FIG. 6). FIG. 6 (a) shows the mRNA expression of atrial natriuretic peptide (ANP), FIG. 6 (b) shows the mRNA expression of brain natriuretic peptide (BNP), and FIG. 6 (c) shows the mRNA expression of the endogenous HB-EGF (rat HB-EGF). In FIGS. 6 (a) to (c), the horizontal axis indicates the time after introduction of the construct.

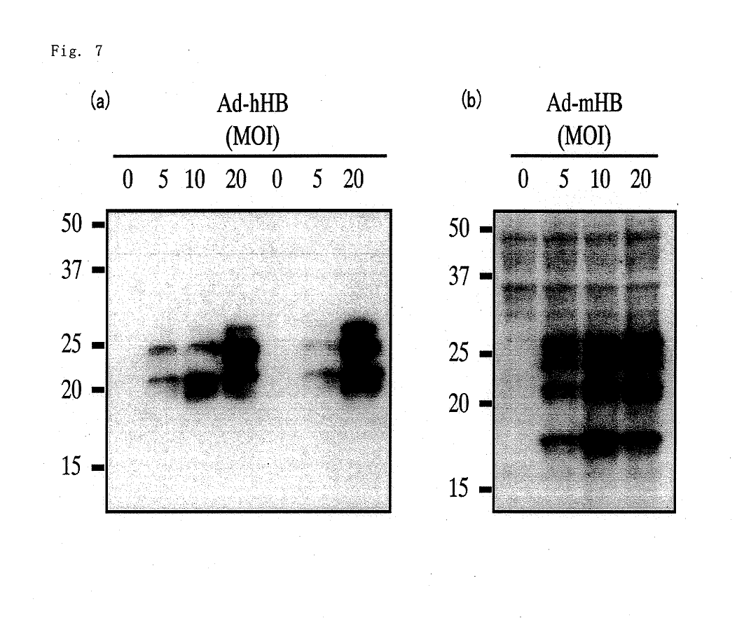

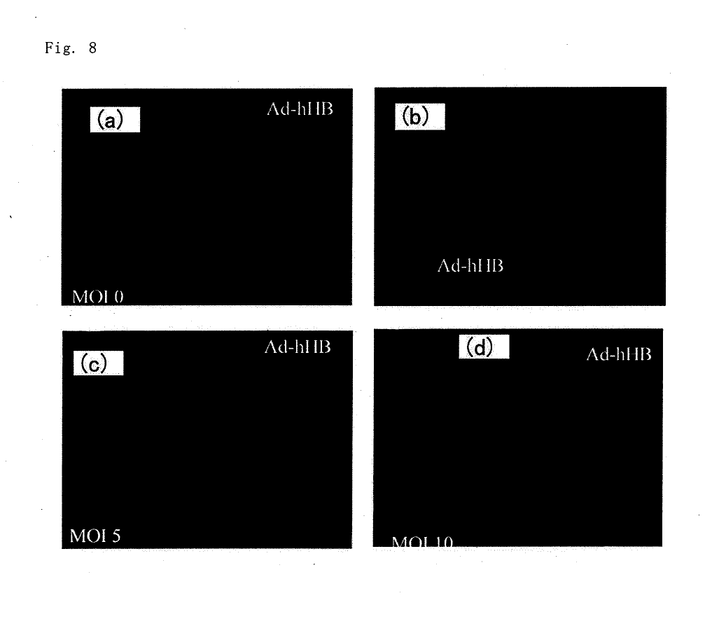

Further, Western blotting was performed 36 hours after infection to determine the expression of the HB-EGF protein in the cultured rat myocardial cells to which Ad-HsHBEGF or Ad-MmHBEGF had been introduced. The results showed that the human HB-EGF gene and the mouse HB-EGF gene were expressed in the protein level in an infection dosage-dependent manner (FIG. 7).

FIG. 7 (a) shows the expression of the human HB-EGF protein 36 hours after infection in the cultured rat myocardial cells detected by Western blotting (nonreducing condition) with an anti-human HB-EGF antibody (monoclonal antibody produced with a hybridoma stored in the Mekada Lab., Department of Cell Biology, Research Institute for Microbial Diseases, Osaka University (3-1, Yamadaoka, Suita, Osaka 565-0871 Japan) (HB-EGF mab clone: #3H4). The anti-human HB-EGF antibody (HB-EGF mab clone: #3H4) can be prepared with the use of a human antigen as the antigen according to the preparation method with the use of a HB^{del/del} mouse described in Mine N, Iwamoto R, Mekada E. HB-EGF promotes epithelial cell migration in eyelid development. Development. 2005 October; 132 (19): 4317-26 (p. 4318). FIG. 7 (b) shows the expression of the mouse HB-EGF protein in the cultured rat myocardial cells detected by Western blotting (nonreducing condition) with an anti-mouse HB-EGF antibody (M-18, made by Santa Cruz, SC1414).

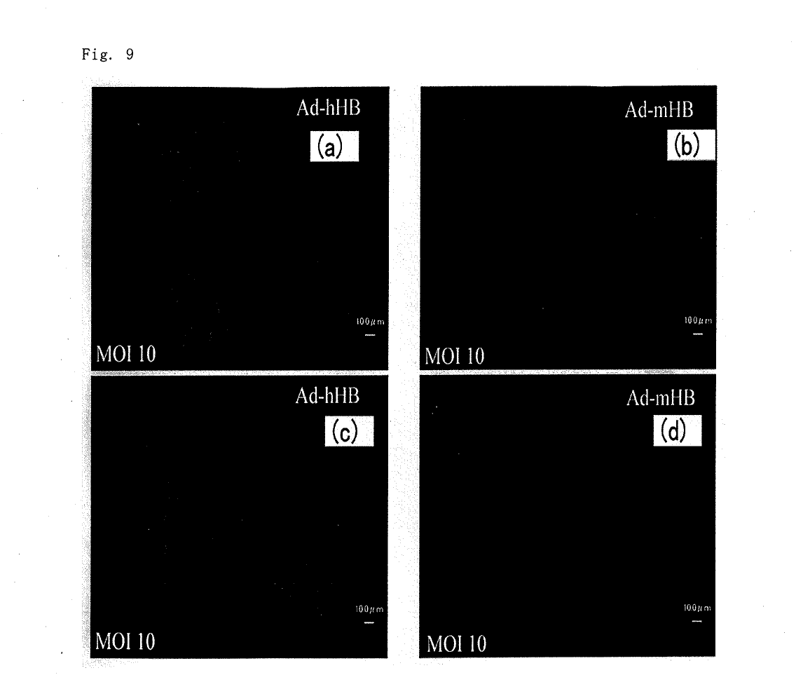

The dose-dependency of the expression of human HB-EGF in cultured myocardial cells and the intracellular localization thereof were examined (FIG. 8). FIGS. 8 (a) to (d) show the results of immunostaining of membrane-bound HB-EGF on the cultured rat myocardial cells into which Ad-HsHBEGF was introduced at (a): MOI=0, (b): MOI=2, (c): MOI=5, and (d): MOI=10, with the use of an anti-human HB-EGF antibody (HB-EGF mab clone: #3H4). In the figures, membrane-bound human HB-EGF portion is stained and shown in white. FIGS. 8 (a) to (d) reveal that the human HB-EGF protein was expressed in an infection dosage-dependent manner on the cell membrane in the cultured rat myocardial cells.

(III) Analysis and Examination of Expression in Cultured Myocardial Fibroblasts from Newborn Rats

Cultured myocardial fibroblasts from newborn rats (Wistar rat (made by Kiwa Laboratory Animals Co., Ltd.)) were used instead of the cultured rat myocardial cells in (II) for infection with the adenovirus construct (Ad-HsHBEGF or Ad-MmHBEGF) prepared above at MOI=10. The localization of the HB-EGF expression in the cultured myocardial fibroblasts was investigated in the same manner as in the above (II).

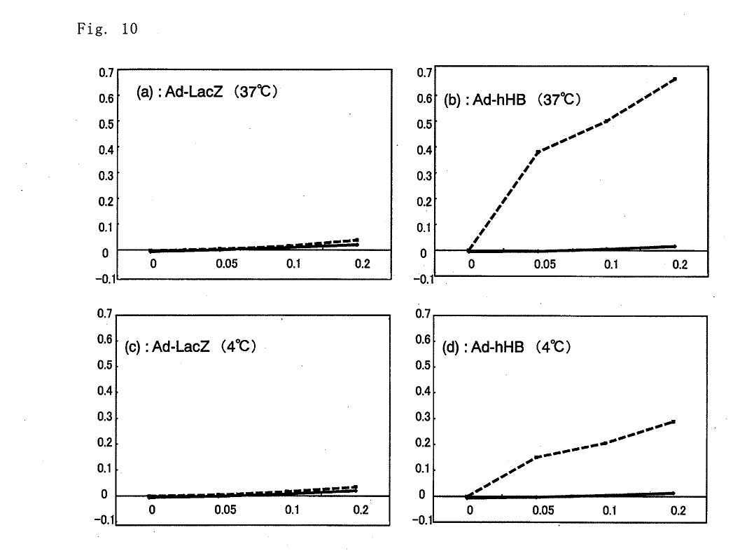

Examination of the HB-EGF localization in the cultured myocardial fibroblasts revealed that the human HB-EGF and the mouse HB-EGF are localized on the cell membrane also in cultured myocardial fibroblasts from newborn rats where the HB-EGF was forcibly expressed (FIG. 9, scale bar: 100 μm). FIGS. 9 (a) and (c) show the results of immunostaining of the cultured myocardial fibroblasts where the human HB-EGF is expressed by infection with Ad-HsHBEGF, with the use of an anti-human HB-EGF antibody (HB-EGF mab clone: #3H4). FIGS. 9 (b) and (d) show the results of immunostaining of the cultured myocardial fibroblasts where the mouse HB-EGF is expressed by infection with Ad-MmHBEGF, with the use of an anti-human HB-EGF antibody (HB-EGF mab clone: #3H4). In the figures, membrane-bound HB-EGF portion is stained and shown in white. Since the HB-EGF mab clone: #3H4 does not detect (bind to) the mouse HB-EGF, the mouse HB-EGF is not stained. It was confirmed that the human HB-EGF expressed by the introduced adenovirus vector was localized on the cell membrane also in cultured myocardial fibroblasts.

In the experiments on uptake and adhesion of liposomes by/to cells shown below, the cultured rat myocardial cells expressing human HB-EGF prepared above were used. As a control, cultured rat myocardial cells into which Ad-LacZ had been introduced were used.

(IV) Experiments on Uptake and Adhesion of Liposomes into/to Cells

In the same manner as in Example 1, whether the Fab′ anti-HB-EGF antibody-bound liposomes adhere to cells and whether the liposomes are taken by cells were investigated at 37° C. or 4° C. The Fab′ anti-HB-EGF antibody-bound liposomes used were the fluorescently-labeled anti-HB-EGF antibody-bound liposomes prepared in Example 1.

The results are shown in FIGS. 10 (a) to (d). The vertical axis of the graph indicates the fluorescence intensity (Ex: 549 nm, Em: 592 nm) corrected for the protein amount. In FIGS. 10 (a) to (d), the dashed line represents a case where PEG liposomes modified with the Fab′ anti-HB-EGF antibody were used, and the solid line represents the case where PEG liposomes not modified with the Fab′ anti-HB-EGF antibody were used. FIG. 10 (a) shows the adhesion and the uptake of the liposomes at 37° C. by the cultured rat myocardial cells into which Ad-LacZ had been introduced, and (b) shows the adhesion and the uptake of the liposomes at 37° C. by the cultured rat myocardial cells into which Ad-HsHBEGF had been introduced. FIG. 10 (c) shows the adhesion and the uptake of the liposomes at 4° C. by the cultured rat myocardial cells into which Ad-LacZ had been introduced, and (d) shows the adhesion and the uptake of the liposomes at 4° C. by the cultured rat myocardial cells into which Ad-HsHBEGF had been introduced.

In the cultured rat myocardial cells into which Ad-LacZ had been introduced, as for both the Fab′ anti-HB-EGF antibody-bound PEG liposomes and the PEG liposomes not modified with the Fab′ anti-HB-EGF antibody, adhesion to and uptake by the cells hardly occurred (FIGS. 10 (a) and (c)). In the cultured rat myocardial cells where the human HB-EGF was expressed by introduction of Ad-HsHBEGF, adhesion and uptake of the Fab′ anti-HB-EGF antibody-bound liposomes were observed at 4° C. and 37° C. Measured values at 37° C. were higher than those at 4° C., suggesting uptake into the cells (FIGS. 10 (b) and (d)). As for the PEG liposomes not modified with the Fab′ anti-HB-EGF antibody, adhesion and uptake into the cells were not observed regardless of the temperature, suggesting the specificity of the anti-HB-EGF antibody to HB-EGF.

### Example 3

Whether the Fab′ anti-HB-EGF antibody-bound PEG liposomes are taken into a breast cancer cell line (MDA-MB-231 cells) was investigated. The fluorescently-labeled anti-HB-EGF antibody-bound liposomes used were those prepared in Example 1.

MDA-MB-231 cells (made by ATCC) were seeded into wells of a 24-well plate (3.0×10⁴ cells/well). To this, a Leibovits L-15 culture medium (containing 10% fetal bovine serum (FBS), 100 units/mL penicillin, and 100 μg/mL streptomycin) (purchased from GIBCO) was added (500 μL/well). The cells were incubated at 37° C. for 4 hours. Subsequently, the culture medium was removed from each well of the plate, a Leibovits L-15 culture medium containing the fluorescently-labeled anti-HB-EGF antibody-bound liposome (HSPC/Chol/DSPE/DSPE-PEG-mal/DiI) prepared in Example 1 or the fluorescently-labeled liposome (HSPC/Chol/DSPE/DiI) as a control (containing 10% fetal bovine serum (FBS), 100 units/mL penicillin, and 100 μg/mL streptomycin) (liposome concentration: 1 mM) in an amount of 25, 50, and 100 μL per well was added to wells so that the liposome concentrations in the samples were 0.05, 0.1, and 0.15 mM, respectively. The prepared samples containing MDA-MB-231 cells (made by ATCC) and the fluorescently-labeled anti-HB-EGF antibody-bound liposomes (or control liposomes) were incubated at 37° C. for 4 hours.

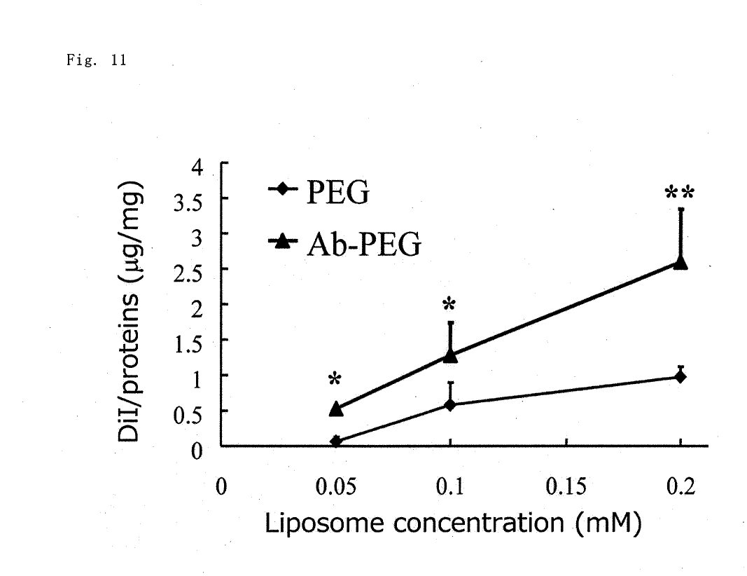

After the cells were washed, the amount of liposomes taken into the MDA-MB-231 cells was determined by fluorescence analysis as in Example 1. The results are shown in FIG. 11. The vertical axis indicates the amount of the liposomes taken into the MDA-MB-231 cells as the amount (μg/mg) of a fluorochrome (octadecylindocarbocyanine (Dil₁₈)) per 1 mg of cellular proteins. The horizontal axis indicates the concentration (mM) of liposomes added to the MDA-MB-231 cells. In the graph, the lozenge represents the control liposome and the triangle represents the anti-HB-EGF antibody-bound liposome. FIG. 11 shows numerical data (measured values) and standard deviations. The asterisk (*) is used to show significant difference (* is p<0.05 and ** is p<0.01). The t-test was used to determine whether there was a significant difference.

As FIG. 11 clearly shows, the uptake of the anti-HB-EGF antibody-bound liposomes by the MDA-MB-231 cells largely surpassed the uptake of the control liposomes.

### Example 4

An intracellular target gene knockdown experiment with the use of the anti-HB-EGF antibody-bound liposomes containing siRNA.

### (I) Preparation of Cells Forced to Express Luciferase

A luciferase gene was introduced into the Vero-H cells prepared in Test Example 1 for forcible expression. Specifically, the Vero-H cells were seeded into wells of a 96-well plate (NUNC) (5.0×10³ cells/0.2 mL per well) and incubated in an atmosphere of 5% carbon dioxide at 37° C. for 24 hours. Then, plasmid DNA pCAG-luc3 (purchased from Nippon Gene) expressing luciferase was introduced into the cells (2 μg per well) with the use of Lipofectamine 2000 (Invitrogen Co.). Incubation was performed in an atmosphere of 5% carbon dioxide at 37° C. for 24 hours.

(II) Preparation of Anti-HB-EGF Antibody-Bound Liposomes Containing siRNA and Control Liposomes

Anti-HB-EGF antibody-bound liposomes containing siRNA for the luciferase gene (sense strand: CUU ACG CUG AGU ACU UCG ATT (SEQ ID NO: 3), antisense strand: UCG AAG UAC UCA GCG UAA GTT (SEQ ID NO: 4) (both made by Hokkaido System Science Co., Ltd.)) were prepared. Specifically, a lipid membrane having a composition ratio of dioleoyl phosphatidylethanolamine (DOPE)/dimyristoyl phosphatidylglycerol (DMPG)/cholesterol=9/2/2 (molar ratio) was produced. A complex solution of siRNA and protamine was separately produced and used to hydrate the lipid membrane. Then, ultrasonication was performed at 37° C. for 10 minutes to give liposomes containing siRNA (2 nmol of siRNA and 80 μg of protamine relative to 10 μmol of the total lipid). Subsequently, 9% of PEG2000 and 1.5% of PEG maleimide 2000 relative to the total lipid were added to modify the liposomes containing siRNA, and then the modified liposomes and Fab′ anti-HB-EGF (the same as prepared in Example 1) in the same amount as maleimide were incubated for 20 hours to be reacted. After the reaction, gel filtration was performed to give purified liposome fractions.

As a control, PEG-liposomes containing the above siRNA were prepared. Specifically, the PEG-liposomes were prepared in the same manner as above except that the modification steps with PEG maleimide 2000 and the Fab′ anti-HB-EGF antibody were skipped.

The amount of siRNA contained in the anti-HB-EGF antibody-bound liposomes and the control liposomes were 5 pmol or 50 pmol.

(III) Luciferase Gene Knockdown by siRNA-Containing Anti-HB-EGF Antibody-Bound Liposomes and by siRNA-Containing Control Liposomes

To the Vero-H cells forced to express luciferase prepared above, the siRNA-containing anti-HB-EGF antibody-bound liposomes or the siRNA-containing control liposomes prepared in (II) were added.

The cell culture medium was replaced with 150 μL/well of a serum-free MEM culture medium containing non-essential amino acids, and 50 μL/well of a diluted solution of the anti-HB-EGF antibody-bound liposomes containing siRNA in RNase-free water (liposome concentration (total lipid concentration): 1 mM for 5 pmol of siRNA and 10 mM for 50 pmol of siRNA) was added. After the addition, the cells were incubated in an atmosphere of 5% carbon dioxide at 37° C. for 24 hours.

The expression of the luciferase gene was investigated by measurement of luciferase activity. The viable cell count was calculated with the use of Luciferase assay CellTiter-Fluor (registered trademark) Cell Viability Assay (Promega). Specifically, 5 μL of the substrate in the kit was added to 1 mL of the buffer solution in the kit, and the mixture was mixed with a vortex to give a Cell Titer reagent. The cell culture medium was replaced with 80 μL of a serum-free MEM culture medium containing non-essential amino acids, and 20 μL of the Cell Titer reagent was added. After mixed for 30 seconds, the mixture was incubated at 37° C. for 30 minutes. Then, the fluorescence intensity was determined with a fluorophotometer (Ex: 400 nm, Em: 505 nm) to calculate the viable cell count. Subsequently, luciferase activity was determined with the use of One-Glo (registered trademark) Luciferase Assay System (Promega). The luciferin substrate reagent in an amount of 100 μL per well was added, and in 3 minutes, the luminescence intensity was measured. The luciferase activity was corrected for the viable cell count.

Luciferase gene knockdown (gene expression inhibition) was evaluated based on the luciferase activity of the Vero-H cells forced to express luciferase without addition of liposomes (set at 100%). The fall of luciferase activity means that the expression of the luciferase gene was inhibited.

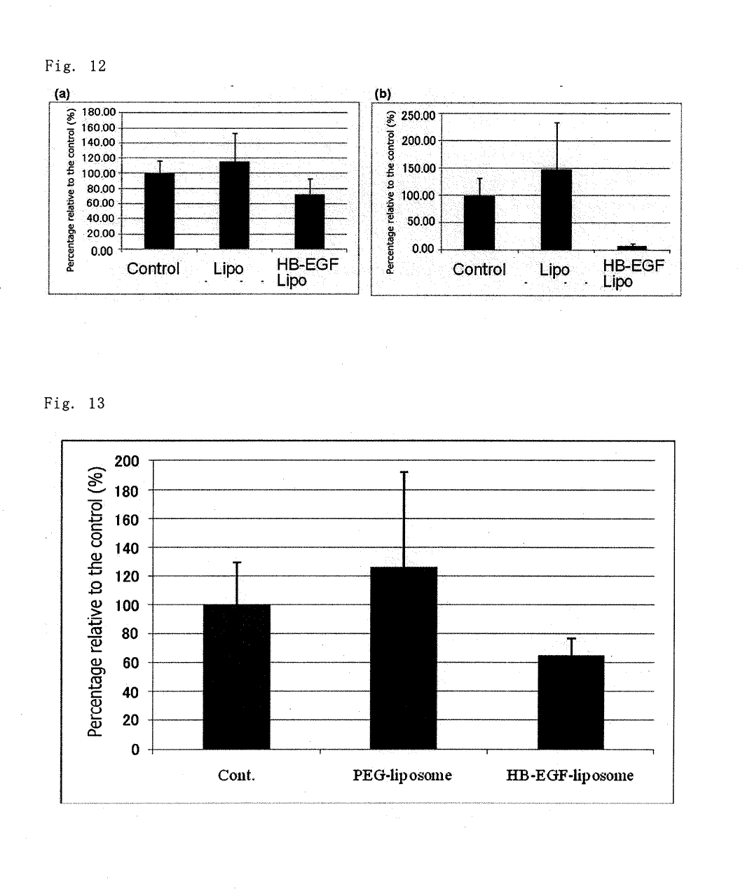

The results are shown in FIGS. 12 (a) and (b). FIG. 12 (a) shows the result of luciferase gene knockdown with the use of the anti-HB-EGF antibody-bound liposomes each containing 5 pmol of siRNA, and the control liposomes. FIG. 12 (b) shows the result of luciferase gene knockdown with the use of the anti-HB-EGF antibody-bound liposomes each containing 50 pmol of siRNA, and the control liposomes.

In FIGS. 12 (a) and (b), “Control” means the Vero-H cells forced to express luciferase (without addition of liposomes). “Lipo” means the Vero-H cells to which the control liposomes containing siRNA were added. “HB-EGF Lipo” means the Vero-H cells to which the anti-HB-EGF antibody-bound liposomes containing siRNA were added. In FIGS. 12 (a) and (b), the vertical axis indicates the luciferase activity relative to the luciferase activity of the “Control” set at 100%.

As FIG. 12 clearly shows, the exogenous highly-expressing gene (luciferase gene) was knocked down by the anti-HB-EGF antibody-bound liposomes containing siRNA. The anti-HB-EGF antibody-bound liposomes containing siRNA inhibited the expression of luciferase more strongly than the control liposomes containing siRNA. Use of the anti-HB-EGF antibody-bound liposomes containing siRNA inhibited the expression of luciferase in a siRNA dosage-dependent manner. Therefore, in the cells highly expressing HB-EGF, the target gene was efficiently knocked down by the anti-HB-EGF antibody-bound liposomes.

### Example 5

Anti-HB-EGF antibody modified liposomes were prepared in the same manner as in Example 4 (II) except that the siRNA to be contained was changed to a siRNA targeting lamin A/C (target sequence: 5′-CTGGACTTCCAGAAGAACA-3′ (SEQ ID NO: 5) B-Bridge International, Inc.). Knockdown of lamin A/C, which is an endogenous gene, was determined by measurement of the amount of mRNA thereof.

The Vero-H cells (3.0×10⁵ cells) were seeded in a 60 mm-dish and preincubated in 5 mL of a MEM culture medium containing non-essential amino acids (solution containing 10% fetal bovine serum (FBS), 100 units/mL penicillin, 100 μg/mL streptomycin and 1 μg/mL geneticin) for 24 hours. The cell culture medium was replaced with 4.5 mL of a serum-free MEM culture medium containing non-essential amino acids, and 500 μL of a diluted solution of the anti-HB-EGF antibody-bound liposomes containing siRNA in RNase-free water (750 μmol of siRNA) was added. Then, the mixture was incubated in an atmosphere of 5% carbon dioxide at 37° C. for 20 hours for introduction of the siRNA into cells. Subsequently, total RNA was extracted with the use of RNeasy Plus Mini Kit (QIAGEN), and after the amount of the total RNA of each sample was equalized, purification of cDNA was performed with the use of T-Primed First-Strand Kit (Amersham Bioscience). The mRNA amounts of lamin A/C and β-actin were determined by real-time PCR, and the amount of mRNA of lamin A/C was corrected using the amount of mRNA of β-actin for determination of the knockdown efficiency of lamin A/C.

The results are shown in FIG. 13. In FIG. 13, “Cont.” means the Vero-H cells to which liposomes were not added.

“Peg-liposome” means the Vero-H cells to which control liposomes containing siRNA were added. “HB-EGF liposome” means the Vero-H cells to which the anti-HB-EGF antibody-bound liposomes containing siRNA were added. In FIG. 13, the vertical axis indicates the amount (%) of lamin A/C mRNA relative to the amount of lamin A/C mRNA in the “Control” set at 100%.

As FIG. 13 clearly shows, lamin A/C which is an endogenous gene was efficiently knocked down by the anti-HB-EGF antibody-bound liposomes containing siRNA. The anti-HB-EGF antibody-bound liposomes containing siRNA inhibited the expression of lamin A/C more strongly than the control liposomes containing siRNA. Therefore, in the cells highly expressing HB-EGF, the target gene was efficiently knocked down by the anti-HB-EGF antibody-bound liposomes.

### Example 6

The inhibitory effect of the anti-HB-EGF antibody-bound liposomes containing doxorubicin on tumor growth in mice to which human breast cancer cells (MDA-MB-231 cells) were transplanted.

### (I) Preparation of Human Breast Cancer Cell (MDA-MB-231 Cell)-Transplanted Mice

MDA-MB-231 cells were purchased from ATCC. The MDA-MB-231 cells were cultured in a Leibovitz L-15 culture medium at 37° C. To the ventral part of 8-week-old Balb/c (nu/nu) mice (purchased from Japan SLC, Inc.), 1.0×10⁷ cells each of the cultured MDA-MB-231 cells were hypodermically transplanted. After transplantation, the mice were kept in conditions of constant temperature and humidity (22° C., 55%) for 12 days and then used for the following experiments.

### (II) Preparation of Anti-HB-EGF Antibody-Bound Liposomes Containing Doxorubicin and of Control Liposomes

Anti-HB-EGF antibody-bound liposomes containing doxorubicin were prepared in the same manner as in Example 4 except that doxorubicin (general name: made by Kyowa Hakko) instead of siRNA was made to be contained in the liposome portion of the liposomes. The amount of doxorubicin contained in the liposomes was 0.26 mol relative to 1 mol of HSPC.

For comparison, anti-HB-EGF antibody-bound liposomes (anti-HB-EGF antibody-bound liposomes not containing doxorubicin) were prepared in the same manner as above except that doxorubicin inclusion was not performed.

As a control, PEG-liposomes containing doxorubicin were prepared in the same manner as in Example 4 except that doxorubicin was used instead of siRNA to be contained in the liposome portion of the PEG-liposomes. The amount of doxorubicin contained in the liposomes was 0.26 mol relative to 1 mol of HSPC.

### (III) Inhibitory Effect of Anti-HB-EGF Antibody-Bound Liposomes Containing Doxorubicin and of Control Liposomes on Tumor Growth

The anti-HB-EGF antibody-bound liposomes containing doxorubicin prepared in (II) were administered to the human breast cancer cell (MDA-MB-231 cell)-transplanted mice (n=5) prepared in (I) by tail vein injection so that the single doxorubicin dose was 10 mg per kg of body weight. For comparison, the anti-HB-EGF antibody-bound liposomes not containing doxorubicin or the control liposomes were administered in the same manner (n=5 each). The liposomes were administered in the form of a suspension in saline. The tumor volume was measured at the start of administration (before therapy) and 48 hours after the administration. The volume of the tumor 48 hours after the administration relative to the volume at the start of administration (set at 100%) was used to evaluate the inhibitory effect on tumor growth. The tumor volume was calculated by the following formula using the smallest diameter and the longest diameter of the tumor measured with a vernier calipers.

Tumor volume=0.4×a×b²

(a: longest diameter of the tumor, b: smallest diameter of the tumor)

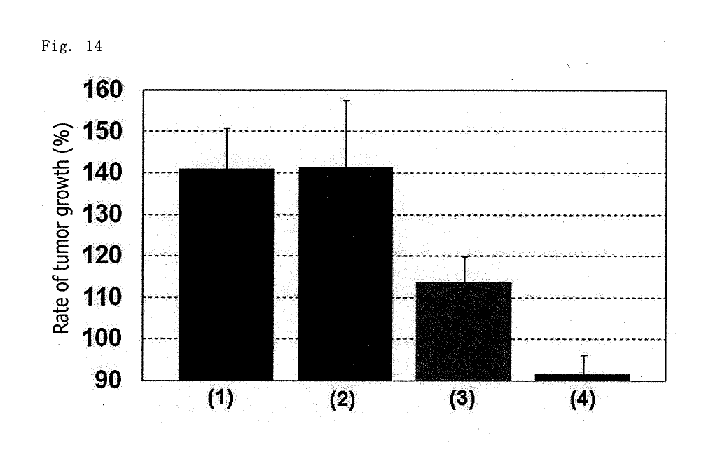

The inhibitory effect 48 hours after the administration is shown in FIG. 14. In FIG. 14, the vertical axis represents the tumor volume 48 hours after the administration (rate of tumor growth) relative to the volume at the start of administration set at 100%. FIG. 14 (1) shows human breast cancer cell-transplanted mice to which neither doxorubicin nor liposome was administered. FIG. 14 (2) shows human breast cancer cell-transplanted mice to which anti-HB-EGF antibody-bound liposomes not containing doxorubicin were administered. FIG. 14 (3) shows human breast cancer cell-transplanted mice to which control liposomes containing doxorubicin were administered. FIG. 14 (4) shows human breast cancer cell-transplanted mice to which anti-HB-EGF antibody-bound liposomes containing doxorubicin were administered.

As FIG. 14 clearly shows, the anti-HB-EGF antibody-bound liposomes containing doxorubicin (4) significantly inhibited tumor growth as compared to the control liposomes containing doxorubicin (3). It has been reported that use of PEG-liposomes containing doxorubicin (the control liposomes (3) in FIG. 14) for an animal model exhibits a higher tumor growth inhibitory effect as compared to use of doxorubicin as it is (S K Huang et al., Cancer Res. 52 (1992), 6774-6781). Regarding this, it was shown that anti-HB-EGF antibody-bound liposomes containing doxorubicin exhibit a significantly higher tumor growth inhibitory effect as compared to doxorubicin directly administered as it is. In the human breast cancer cell-transplanted mice to which the anti-HB-EGF antibody-bound liposomes not containing doxorubicin were administered, tumor growth was to the same degree as in the control was observed.

### Example 7

Myocardial cells were prepared in the same manner as in the experiment with the use of cultured rat myocardial cells in Example 2 (II). Inhibitory effect on gene expression can be examined by addition of the anti-HB-EGF antibody-bound liposomes containing siRNA to the myocardial cells.

Anti-HB-EGF antibody modified liposomes were prepared in the same manner as in Example 5 except that the siRNA to be contained was changed to a siRNA targeting lamin A/C (target sequence: 5′-GGTGGTGACGATCTGGGCT-3′ (SEQ ID NO: 6) B-Bridge International, Inc.). Knockdown of lamin A/C, which is an endogenous gene, can be determined by measurement of the amount of mRNA thereof.

Cultured myocardial cells from newborn rats, Wistar rat, (made by Kiwa Laboratory Animals Co., Ltd.) were seeded into wells of a 6-well plate (1.5×10⁶ cells/well). To this, one of the Ad-HsHBEGF, Ad-MmHBEGF, and Ad-LacZ prepared in Example 2 (I) was added and mixed to infect the cultured rat myocardial cells in the same manner as in Example (II). Into the cultured rat myocardial cells, 36 hours after infection, the anti-HB-EGF antibody modified liposomes containing siRNA to lamin A/C were introduced. By real-time PCR performed 24 hours after the liposome introduction to determine the lamin A/C mRNA expression in cultured rat myocardial cells, knock out of the gene can be examined. 48 hours after the liposome introduction, expression inhibition of lamin A/C protein can also be confirmed by Western blotting. The anti-HB-EGF antibody-bound liposomes containing siRNA more efficiently knock down lamin A/C in cultured rat myocardial cells infected with Ad-HsHBEGF than in cultured rat myocardial cells infected with Ad-LacZ.

The results of the above Examples confirmed that HB-EGF-bound, protein complexes, such as an anti-HB-EGF antibody-bound liposome, can deliver a drug, siRNA, etc. specifically to failing heart (pressure overload heart, heart after infarction, etc.), cancer cell, or the like where increased expression of HB-EGF is observed. Therefore, it was revealed that the anti-HB-EGF antibody-bound liposomes are useful as a novel therapeutic system targeting such cardiovascular diseases as failing heart and arteriosclerosis site, gynecological cancers, etc. In particular, since no drug delivery system (DDS) intended for cardiac failure has been reported so far, this new system is revolutionary. Typical models of cardiac failure in which anti-HB-EGF antibody-bound liposomes can be used include clinical conditions, such as myocarditis (autoimmune, infectious, etc.), cardiac infraction, and ischemia-reperfusion, in which increased expression of HB-EGF accompanied by inflammation easily affecting vascular endothelium is observed. Further, it is expected that HB-EGF-bound protein complexes, such as an anti-HB-EGF antibody-bound liposome, are selectively delivered to target cells expressing HB-EGF and, after bound to the target cells, promote uptake of the HB-EGF into the cells, thereby inhibiting cell growth induced by the growth factor HB-EGF. Therefore, in cancer cells, arteriosclerosis sites, and the like, unique tumor growth inhibitory effect that other DDS technologies do not have can be expected. Further, it is known that increased expression of HB-EGF in cancer tissue is observed, not only in cancer cells but also in endothelial cells of neovascular vessels. Therefore, DDS targeting HB-EGF efficiently functions also as DDS targeting neovascular vessels in an affected part by cancer, arteriosclerosis, or the like.

### INDUSTRIAL APPLICABILITY

The present invention enables efficient introduction of an object substance into target cells, such as myocardial cells, and cancer cells. Therefore, the present invention is useful in the fields of medical treatment and research.

1-10. (canceled)

11. A complex essentially comprising a protein having an action of binding to a membrane-bound HB-EGF and of thereby promoting the uptake of the membrane-bound HB-EGF by cells, and a carrier.

12. The complex according to claim 11, wherein the protein is an anti-HB-EGF antibody or a fragment thereof.

13. The complex according to claim 11, wherein the protein is a diphtheria toxin mutant CRM197 or a fragment thereof.

14. The complex according to claim 11, wherein an object substance to be introduced into cells is enclosed in the carrier, or the object substance forms a complex together with the carrier.

15. The complex according to claim 14, wherein the object substance is a drug for cancer, cardiac failure, nerve disease, or pulmonary disease, or a nucleic acid.

16. A therapeutic or prophylactic agent for cancer, cardiac failure, nerve cell disease, or pulmonary disease, in which HB-EGF is highly expressed, the agent comprising a complex essentially comprising a protein having an action of binding to a membrane-bound HB-EGF and of thereby promoting the uptake of the membrane-bound HB-EGF by cells, and a carrier.

17. A diagnostic agent for cancer, cardiac failure, nerve cell disease, or pulmonary disease, in which HB-EGF is highly expressed, the agent comprising a complex essentially comprising a protein having an action of binding to a membrane-bound HB-EGF and of thereby promoting the uptake of the membrane-bound HB-EGF by cells, and a carrier.

18. A promoter for introduction of an object substance into cells, the agent comprising a complex essentially comprising a protein having an action of binding to a membrane-bound HB-EGF and of thereby promoting the uptake of the membrane-bound HB-EGF by cells, and a carrier.

19. A method for promoting introduction of an object substance into cells, the method comprising a step of adding a complex essentially comprising a protein having an action of binding to a membrane-bound HB-EGF and of thereby promoting the uptake of the membrane-bound HB-EGF by cells, and a carrier; and an object substance to be introduced into cells, to cells which express HB-EGF or a step of administering the complex and the object substance to an animal other than a human.

20. A kit comprising a complex essentially comprising a protein having an action of binding to a membrane-bound HB-EGF and of thereby promoting the uptake of the membrane-bound HB-EGF by cells, and a carrier.

21. A therapeutic or prophylactic agent for breast cancer, the agent comprising a complex essentially comprising an anti-HB-EGF antibody or a fragment thereof having an action of binding to a membrane-bound HB-EGF and of thereby promoting the uptake of the membrane-bound HB-EGF by cells, and a liposome in which an anticancer drug is enclosed.

22. A therapeutic or prophylactic agent for cardiac failure, the agent comprising a complex essentially comprising an anti-HB-EGF antibody or a fragment thereof having an action of binding to a membrane-bound HB-EGF and of thereby promoting the uptake of the membrane-bound HB-EGF by cells, and a carrier in which a drug for cardiac failure is enclosed.

23. A promoter for introduction of a drug for cardiac failure into failing myocardial cells, the promoter comprising a complex essentially comprising an anti-HB-EGF antibody or a fragment thereof having an action of binding to a membrane-bound HB-EGF and of thereby promoting the uptake of the membrane-bound HB-EGF by cells, and a carrier.

24. A promoter for introduction of a nucleic acid into failing myocardial cells, the promoter comprising a complex essentially comprising an anti-HB-EGF antibody or a fragment thereof having an action of binding to a membrane-bound HB-EGF and of thereby promoting the uptake of the membrane-bound HB-EGF by cells, and a carrier in which siRNA is enclosed.
